(19) United States
(12) Patent Application Publication
(10) Pub. No.: US 2022/0112303 A1
(43) Pub. Date: Apr. 14, 2022

(54) ANTI-CD24 ANTIBODY AND USES THEREOF

(71) Applicant: Ichilov Tech Ltd., Tel-Aviv (IL)

(72) Inventors: Nadir ARBER, Tel-Aviv (IL); Shiran SHAPIRA, Petach-Tikva (IL); Diana KAZANOV, Rishon-LeZion (IL)

(73) Assignee: Ichilov Tech Ltd., Tel-Aviv (IL)

(21) Appl. No.: 17/560,582

(22) Filed: Dec. 23, 2021

Related U.S. Application Data
(63) Continuation of application No. PCT/IL2020/050716, filed on Jun. 25, 2020.
(60) Provisional application No. 62/866,008, filed on Jun. 25, 2019.

(51) Int. Cl. C07K 16/28; A61P 35/00

(52) U.S. Cl. CPC C07K 16/2896; C07K 16/2809; C07K 2317/31; C07K 2317/565; C07K 2317/24; A61P 35/00

(57) ABSTRACT

An anti-CD24 antibody is provided. Accordingly, there is provided an antibody comprising an antigen recognition domain which specifically binds CD24 and comprises complementarity determining regions (CDRs) as set forth in SEQ ID NOs: 2, 3 and 4 arranged in a sequential order from N to C on a light chain of the antibody and CDRs as set forth in SEQ ID NOs: 6, 7 and 8 arranged in a sequential order from N to C on a heavy chain of said antibody. Also provided are polynucleotides encoding the antibody, host cells expressing the antibody the uses thereof.

### RELATED APPLICATIONS

This application is a Continuation of PCT Patent Application No. PCT/IL2020/050716, having international filing date of Jun. 25, 2020 which claims the benefit of priority under 35 USC § 119(e) of U.S. Provisional Patent Application No. 62/866,008 filed on Jun. 25, 2019. The contents of the above applications are all incorporated by reference as if fully set forth herein in their entirety.

### SEQUENCE LISTING STATEMENT

The ASCII file, entitled 90526SequenceListing.txt, created on Dec. 23, 2021, comprising 111,644 bytes, submitted concurrently with the filing of this application is incorporated herein by reference.

### FIELD AND BACKGROUND OF THE INVENTION

The present invention, in some embodiments thereof, relates to an anti-CD24 antibody and uses thereof.

Cancer is one of the leading causes of death in the western world. In spite of all biomedical advances and the efforts undertaken by society, public institutions and the pharmaceutical and biotech industries to tackle this disease, many malignancies still have a huge mortality rate. As an example, colorectal cancer (CRC) and pancreatic cancer (PC) have high annual incidence rates and poor prognosis. Current treatment modalities include chemotherapy, surgery, gene therapy, immunotherapy, radiation therapy, and combinations of these. Historically, surgery was considered the best treatment for CRC and PC. However, long-term survival following surgery is only moderately achieved due to recurrence in remote sites. Unfortunately, less than 20% of PC patients are suitable for resection and potential cure by the time of their first diagnosis. Specific therapies in patients with advanced CRC or PC have been studied with limited success. Trials evaluating the use of chemotherapy and radiation therapy both alone and in combination have shown only moderate/marginal improvements in survival for CRC or PC, with relatively high profile of side effects. Thus, there remains a need for more effective treatment options for cancer in general and CRC and PC in particular.

CD24, also known as heat-stable antigen (HSA) in mice, is a heavily glycosylated phosphatidylinositol-anchored mucin-like cell-surface protein. Physiologically, the CD24 protein is expressed mainly on hematopoietic subpopulations of B-lymphocytes, various epithelial cells, muscle and neural cells. It plays a crucial role in cell selection and maturation during hematopoiesis and is expressed during the embryonic period, on developing neural and pancreatic cells. In addition, CD24 is a potential ligand for P-selectin which functions as an adhesion molecule that enhances platelets aggregation.

CD24 was shown to be overexpressed in various malignant tissues including colorectal cancer, B-cell lymphomas, gliomas, small-cell and non-small cell lung, hepatocellular, renal cell, nasopharyngeal, bladder, uterine, epithelial ovarian, breast, prostate and pancreatic carcinomas [see e.g. Kristiansen et al. (2004), J Mol Histol. 35(3): 255-62; and Weichert et al. (2005), Clin Cancer Res 11(18): 6574). Moreover, its expression was found to correlate with increased growth rate, motility and survival in carcinoma cell lines derived from several organs [Baumann et al. (2005), Cancer Res. 65: 10783-93; Smith, et al. (2006) Cancer Res. 66: 1917-22] and with a more aggressive course of cancer. Thus, Weichert et al. (2005), found that increased expression of CD24 in the cytoplasm correlates with higher tumor stage, grade and presence of metastasis and concluded that overexpression of CD24 in the cytoplasm is a marker for poorer prognosis. In addition, the role of CD24 in platelet aggregation may explain the involvement with cancer metastases and worse prognosis (Sammar, M., et al., 1994; Aigner, S., et al., 1997; Aigner, S., et al., 1998). To date, several monoclonal antibodies against CD24 have been generated and were shown to inhibit tumor (e.g. CRC and hepatocellular carcinoma) cell growth in-vitro and in-vivo [see e.g. Shapira et al. (2011) Gastroenterology, 140(3):935-46; Sun et al. (2017), Oncotarget. 8(31): 51238-51252].

Additional background art includes International Application Publication Nos: WO2007/088537, WO2008/059491, WO2009/063461, WO2009/074988 and WO2018/216006.

### SUMMARY OF THE INVENTION

According to an aspect of some embodiments of the present invention there is provided an antibody comprising an antigen recognition domain which specifically binds CD24 and comprises complementarity determining regions (CDRs) as set forth in SEQ ID NOs: 2, 3 and 4 arranged in a sequential order from N to C on a light chain of the antibody and CDRs as set forth in SEQ ID NOs: 6, 7 and 8 arranged in a sequential order from N to C on a heavy chain of the antibody.

According to some embodiments of the invention, the light chain amino acid sequence comprises an amino acid sequence as set forth in SEQ ID NO: 1.

According to some embodiments of the invention, the heavy chain amino acid sequence comprises an amino acid sequence as set forth in SEQ ID NO: 5.

According to some embodiments of the invention, the light chain amino acid sequence comprises an amino acid sequence as set forth in SEQ ID NO: 1 and the heavy chain amino acid sequence comprises an amino acid sequence as set forth in SEQ ID NO: 5.

According to some embodiments of the invention, the antibody is a humanized antibody.

According to some embodiments of the invention, the antibody is multispecific.

According to some embodiments of the invention, the antibody is bispecific.

According to some embodiments of the invention, the antibody comprises an antigen recognition domain which specifically binds an immune cell.

According to some embodiments of the invention, the antigen recognition domain which specifically binds the immune cell binds CD3.

According to an aspect of some embodiments of the present invention there is provided a polynucleotide encoding the antibody.

According to an aspect of some embodiments of the present invention there is provided a host cell expressing the antibody.

According to an aspect of some embodiments of the present invention there is provided a method of producing an anti-CD24 antibody, the method comprising:

(a) culturing the cells under conditions which support production of the antibody; and

(b) recovering the antibody.

According to an aspect of some embodiments of the present invention there is provided a method of treating a disease associated with cells expressing CD24 in a subject in need thereof, the method comprising administering to the subject a therapeutically effective amount of the antibody, thereby treating the disease in the subject.

According to some embodiments of the invention, the method further comprising administering to the subject a therapy for treating the disease.

According to an aspect of some embodiments of the present invention there is provided the antibody, for use in treating a disease associated with cells expressing CD24 in a subject in need thereof.

According to some embodiments of the invention, the antibody for use further comprising a therapy for treating the disease.

According to an aspect of some embodiments of the present invention there is provided an article of manufacture comprising the antibody and a therapy for treating a disease associated with cells expressing CD24.

According to some embodiments of the invention, the disease is cancer.

According to some embodiments of the invention, the cancer is selected from the group consisting of colorectal cancer, pancreatic cancer, B-cell lymphomas, glioma, small-cell lung cancer, non-small cell lung, hepatic cancer, renal cancer, nasopharyngeal cancer, bladder cancer, uterine cancer, ovarian cancer, breast cancer and prostate cancer.

According to some embodiments of the invention, the cancer is colorectal cancer or pancreatic cancer.

According to some embodiments of the invention, the therapy is selected from the group consisting of immunotherapy, chemotherapy and radiotherapy.

According to an aspect of some embodiments of the present invention there is provided a method of activating an immune cell towards a cell expressing CD24 in a subject in need thereof, the method comprising administering to the subject a therapeutically effective amount of the antibody, thereby activating the immune cell towards the cell expressing the CD24.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of the humanization of murine immunoglobulin SWA11 V genes.

FIGS. 2A-B show alignment analyses of nucleotide and deduced amino acid sequences of the heavy chain V-region (FIG. 2A, SEQ ID NO: 57-61) and light chain V-region (FIG. 2B, SEQ ID NO: 62-66) of murine SWA11, donor sequences from IGVH-28*02 and IGK4-1*01, human JH6 and JK2, and the humanized SWA11. The changes to be made in SWA11 VH and V-kAPPA in order to humanize them are marked in gray. The SWA11 residues that were kept as they were introduced during the affinity maturation from the 36-60.a1.85 and 8-30 germlines are marked in green. The CDRs are marked in red.

FIG. 3 demonstrates the results of the pharmacokinetic (PK) studies. Female Balb/c mice (n=13) received a single intravenous (i.v) injection of the humanized antibody (referred to herein as HuNS17) at a dose of 5 mg/kg. Blood samples were collected from the periorbital sinus at the following time points: 15 and 30 minutes; 1, 6, and 24 hours; and 3, 7, 9, 14, 19, 21, and 28 days, and transferred into blood collection tubes for serum separation. One of the mice was injected only with PBS and served as negative control. Serum concentrations of the antibody were determined by antigen-based ELISA.

FIG. 4 is a graph demonstrating the binding strength of the humanized anti-CD24 antibody HuNS17 as determined by Biomolecular Interaction Analysis, Biacore, using CD24 antigen captured on the surface of the sensor chip. The different lines represent different concentrations of the analyte (40, 60, 100, 200 and 300 nM).

FIG. 5 is a dose response graph demonstrating cell-based antibody-dependent cell-mediated cytotoxicity (ADCC) of the humanized HuNS17 antibody. In this assay, HT-29 and PANC-1 tumor cells were used as target cells (T); and Fc receptor stabilized NK-92 stable cells (NK-92/CD16a.V/V) were used as the effector cells (E), in a E:T ratio of 10:1. Lysis was evaluated by using LDH kit. Absorbance data was read at OD492 nm and OD650nm. The background (0D650 nm) subtracted OD492 nm data were analyzed to study the LDH release. The percentages of cell lysis were calculated according the following formula:

Cell lysis %=100*(1−(ODSample data−ODtumor cells+NK cells)/(ODMaximum release−ODMinimum release)).

FIGS. 6A-B demonstrate the results of acute intravenous maximum tolerated dose (MTD) evaluation. FIG. 6A is a graph demonstrating the distance travelled follow-up and mobility in the different groups during open field test. No difference was observed between the groups. FIG. 6B demonstrates the clinical hematology and chemistry results. All data obtained was within the accepted range and with no clinical significance.

FIG. 7 shows graphs of in-vivo efficacy evaluation of the un-armed HuNS17 in xenografts models of prostate and pancreatic cancers.

FIG. 8 is a schematic illustration of the different humanized anti-CD24 and anti-CD30 Fab fragments constructed.

FIG. 9 is a graph demonstrating binding of the three generated Fab derivatives as determined by ELISA. Decimal dilutions of the phages were used, 1×10¹²-1×10¹⁰, from each. “H” represents pCOMB3X-H(CD24)L(CD30), “L” represents the pCOMB3X-H(CD30)L(CD24) and “Fab” represents the pCOMB3X-H(CD24)L(CD24). The procedure was performed as described in Benhar et al., (current Protocols in Immunology, 2002).

FIG. 10 is a schematic representation of libraries design:, i.e. designing affinity maturation of Humanized SWA11 : looking for CDR diversity in 200 homologies by IgBlast. The yellow marks represent the modified loci, the positions that were randomized. The others are conserved loci among the different homologies that have been used to design these libraries.

FIG. 11 is a schematic representation of the ELISA for determining potential binders from the library of phage antibodies. Individual colonies after the third and fourth panning cycles were picked into 100 μl YTAG medium in sterile 96-wells plates (Master plate) and grew overnight at 37° C. with shaking (150 rpm). Following, 10 μl from each well were transferred to a second 96-wells plate (Rescue plate) and the rescued phages were used for ELISA. ELISA plates were coated with 100 μl per well of antigen protein overnight at 4° C. at a concentration of 5 μg/ml in PBS. Binding of Fab-displaying phage was detected by horseradish peroxidase (HRP)-conjugated rabbit anti-M13 antibody. ELISA plates were blocked with 3% skim milk. Since affinity selection often results in nonspecific phages being isolated along with the specific ones, binding to an irrelevant antigen (BSA) was always tested in parallel. The master plates were used as the source for the positive monoclonal clones that were identified by the phage ELISA, for further manipulation.

FIGS. 12A-B are schematic representations of mammalian pcDNA4/TO and pcDNA4-CMV-IgL-CMV-IgH expression vectors. Represented are maps of pcDNA4/TO backbone plasmid (FIG. 12A) and pcDNA4-CMV-IgL-CMV-IgH (FIG. 12B). pcDNA4-CMV-IgL-CMV-IgH was used to generate an inducible expression and secretion system for whole IgG humanized anti-CD24 mAbs.

FIGS. 13A-C demonstrate binding of the matured antibody to CD24, as determined by antigen-based ELISA (FIG. 13A), whole cell ELISA (FIG. 13B) and FACS analysis (FIG. 13C). HT29 and HCT116 cells are colorectal cancer cell lines; HT29 cells express CD24 while HCT116 cells express only very low levels of CD24 and served as negative control.

FIG. 14 demonstrates that the matured antibody inhibits the proliferation of breast cancer cells, as demonstrated qualitatively by microscopic observation and quantitatively by the enzymatic MTT assay. BT549 and 468 are triple negative breast cancer cell lines.

FIGS. 15A-D demonstrate specificity, binding strength, stability and ADCC activity of the matured antibodies. FIG. 15A shows the Biacor results of several matured clones compared to the humanized derivative HuNS17 antibody. FIG. 15B shows the results of an in vitro stability test for the matured antibody. Briefly, the purified mAb was diluted in PBS to a final concentration of 1 μg/ml. The antibody was then incubated at 37° C. and in each time point, as indicated in the graph, sample was taken from the tested item and kept in 4° C. At the end, the antibody was analyzed for its activity by an antigen-based ELISA. FIG. 15C demonstrate the ADCC activity of the matured antibody, as determined by an E/T optimization assays on HT29 tumor cell line. Target cells were pre-incubated with 10 μg/ml of NS17 for 30 min at 37° C./5% incubator. PBMCs were added to initiate the ADCC effects at 3 different E/T ratios. Following incubation at 37° C./5% CO2 incubator for 6 hours, cell supernatant was collected for measuring released LDH to calculate % target cell lysis. Herceptin mediated ADCC lysis of MCF-7 cells was used as a positive control. FIG. 15D demonstrate the ability of matured antibody NS17 to recognize and specifically bind to CD24 by whole-cell ELISA. CD24-positive cells (HT29, CRC; colo257 and Panc-1, pancreas; sh-sy5y, neuroblastoma) and CD24-negative cells (HCT116, CRC) were used.

FIGS. 16A-B demonstrate the library design. FIG. 16A is a schematic representation of a design of a library based on the sequences of the binders that were isolated in the first maturation process in order to find additional and possibly better binders. The numbers (in purple and green) represent the length of the primers that were used. The red numbers represent that number of the primers. The designed library comprised CDR1 and CDR2 mutations and is illustrated in FIG. 16B.

FIG. 17 demonstrates the VL and VH sequences of the humanized matured NS17 anti-CD24 antibody (SEQ ID NOs: 1 and 5), as determined by the method of Kabat et al. The CDRs (SEQ ID Nos: 2, 3, 4, 6, 7 and 8) are highlighted in yellow.

FIG. 18 shows the expression pattern of CD24 in normal human tissues, using an FDA-approved normal human organ tissue microarray. 32 types of normal human organs were included based on FDA guidelines, where each organ was taken from 3 normal human individuals. An anti-CD24 antibody was used to detect CD24 expression. The vast majority of the normal tissues showed no expression of CD24. Phechromocytoma sample has been used as a positive control.

FIG. 19 demonstrates that CD24 is highly expressed in most human malignancies. Several TMAs (tumor microarray) have been used to study CD24 expression pattern in various human malignancies.

FIG. 20 demonstrates that the LAP epitope which is recognize by the matured mAb is very unique. A large homology comparison analysis using the Uniprot (www(dot)uniprot(dot)org) was performed. 250 candidates with different percentage of identity were observed. However, in none of them the LAP epitope exist. Several example of the CD24 sequence (SEQ ID NO: 70-74) are shown and this epitope does not appear in any of them.

FIG. 21 demonstrates efficacy of the NS17 antibody in nude mice bearing a colorectal cancer (CRC) xenograft.

FIGS. 22A-C demonstrate the in-vivo efficacy of the humanized matured NS17 anti-CD24 antibody using two patient-derived xenograft (PDX) models. FIG. 22A shows the results of the immunotherapy experiment in humanized PDX model of head and neck cancer. Keytruda-resistant tumor cells were taken from a head and neck patient and injected into irradiated NGS male mice. PBMCs as well as BM were taken from the same patient and were injected to the mice for their “humanization”. The antibody, 1 mg/mouse, was given twice weekly for 3 weeks and tumor volume was measured. FIG. 22B shows CD24 expression level in a primary CR adenocarcinoma (harvest site-sigmoid colon) biopsy taken from a 61 years old female, as determined by IHC. This poorly differentiated tumor expressed high levels of CD24 and was chosen as the donor for an additional PDX study. FIG. 22C shows the results of NS17 mAb as a monotherapy in one Low Passage champions TumorGraft® model of human colorectal cancer expressing CD24 in Humanized mice. Test System-Species: Mouse; Strain: NOD.Cg-Prkdcscid I12rgtm1Sug/JicTac (NOG), Source: Taconic, Gender: Female, Target age at initiation of dosing: 4 weeks of age. Target weight at initiation of dosing: 17 grams. Within 4 hours prior to inoculation of mice with CD34+cord blood cells, female NOG mice were sublethally irradiated with 175 cGy whole body irradiation. Following, CD34+cells were prepared (100,000-120,000 cells/mouse) and injected into the lateral tail vein of irradiated mice. Animals were monitored via clinical observations for three months of humanization period. ˜150 μl of peripheral blood were removed at week 9 and 12 post CD34+ engraftment to assess humanization using mCD45/huCD45/huCD3/huCD19 markers. Tumors were implanted to mice once humanization was confirmed at the 12 weeks post engraftment period.

FIGS. 23A-B show expression of CD24 on the cell surface of human lymphoma Nalm6 cells (FIG. 23A) and expression of CD11b, CD14, CD45, CD64, CD163 and CD206 on the surface of Monocytes Derived Macrophages (MDM) (FIG. 23B), as determined by flow cytometry.

FIG. 24 demonstrate phagocytosis of human lymphoma Nalm6 cells by MDMs induced by the humanized mature NS17 anti-CD24 mAb.

FIGS. 25A-B demonstrate a Bispecific T-cell engager (BiTE) antibody comprising the VH and VL chains of the humanized mature NS17 and the VH and VL chains of an anti-CD3 antibody. FIG. 25A is a schematic representation of an anti-CD24 anti-CD3 BiTE. FIG. 25B shows binding of the generated anti-CD24 anti-CD3 BiTEs, SEQ ID NO: 29 (BiTE 1) and SEQ ID NO: 31 (BiTE 2), to CD24+CD3+human PBMCs, as determined by flow cytometry.

### DESCRIPTION OF SPECIFIC EMBODIMENTS OF THE INVENTION

The present invention, in some embodiments thereof, relates to an anti-CD24 antibody and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Cancer is one of the leading causes of death in the western world. Current treatment modalities include chemotherapy, surgery, gene therapy, immunotherapy, radiation therapy, and combinations of these.

CD24 was shown to be overexpressed in various malignant tissues. Moreover, increased expression of CD24 was found to correlate with tumor stage, grade and presence of metastasis and thus considered a marker for poorer prognosis.

While reducing specific embodiments of the present invention to practice the present inventors have generated a novel humanized affinity matured anti-CD24 antibody, referred to herein as NS17, which showed high specificity, affinity, stability and pharmacokinetic characteristics and was able to induce antibody-dependent cell-mediated cytotoxicity (ADCC), complement dependent cytotoxicity (CDC) and antibody-dependent cellular phagocytosis (ADCP) and inhibit tumor growth in-vitro and in-vivo. Furthermore, the present inventors have generated a bispecific antibody comprising the VH and VL chains of NS17 and the VH and VL chains of an anti-CD3 antibody.

These results place the antibodies as an important clinical tool for the treatment of CD24 associated medical conditions such as cancer.

Thus, according to a first aspect of the present invention, there is provided an antibody comprising an antigen recognition domain which specifically binds CD24 and comprises complementarity determining regions (CDRs) as set forth in SEQ ID NOs: 2, 3 and 4 arranged in a sequential order from N to C on a light chain of the antibody and CDRs as set forth in SEQ ID NOs: 6, 7 and 8 arranged in a sequential order from N to C on a heavy chain of said antibody.

As used herein, the term “CD24” refers to phosphatidylinositol-anchored mucin-like cell-surface protein encoded by the CD24 gene (Gene ID: 100133941). According to specific embodiments, CD24 refer to the human CD24, such as provided in GenBank Accession No. NP_037362.

The term “antibody” as used in this invention includes intact molecules as well as functional fragments thereof (that are capable of binding to an epitope of an antigen).

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv Fv (scFv), a disulfide-stabilized Fv (dsFv), an Fab, an Fab′, and an F(ab′)2.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (see, e.g., Chothia et al., Nature 342: 877-883, 1989.), a compromise between Kabat and Chothia using Oxford Molecular's AbM antibody modeling software (now Accelrys®, see, Martin et al., 1989, Proc. Natl Acad Sci USA. 86: 9268;

and world wide web site www(dot)bioinf-org(dot)uk/abs), available complex crystal structures as defined by the contact definition (see MacCallum et al., J. Mol. Biol. 262: 732-745, 1996) and the “conformational definition” (see, e.g., Makabe et al., Journal of Biological Chemistry, 283: 1156-1166, 2008).

As used herein, the “variable regions” and “CDRs” may refer to variable regions and CDRs defined by any approach known in the art, including combinations of approaches.

According to specific embodiments, the identity of the amino acid residues in the antibody that make up the CDRs, the variable regions, the light chain and/or the heavy chain is determined by the method of Kabat et al. (See, e.g., Kabat et al., 1992, Sequences of Proteins of Immunological

Interest, 5th ed., Public Health Service, NIH, Washington D.C.).

The antibody disclosed herein comprises CDRs as set forth in SEQ ID NOs: 2, 3 and 4 arranged in a sequential order from N to C on a light chain of the antibody and CDRs as set forth in SEQ ID NOs: 6, 7 and 8 arranged in a sequential order from N to C on a heavy chain of said antibody.

According to specific embodiments, the variable region of the light chain (VL) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 1.

Sequence identity or homology can be determined using any protein or nucleic acid sequence alignment algorithm such as Blast, ClustalW, and MUSCLE.

According to specific embodiments, the variable region of the light chain (VL) is as set forth in SEQ ID NO: 1.

Thus, according to specific embodiments, the light chain amino acid sequence comprises an amino acid sequence as set forth in SEQ ID NO: 1.

According to specific embodiments, the variable region of the heavy chain (VH) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 5.

According to specific embodiments, the variable region of the heavy chain (VH) is as set forth in SEQ ID NO: 5.

Thus, according to specific embodiments, the heavy chain amino acid sequence comprises an amino acid sequence as set forth in SEQ ID NO: 5.

According to a specific embodiment, the light chain amino acid sequence comprises an amino acid sequence as set forth in SEQ ID NO: 1 and the heavy chain amino acid sequence comprises an amino acid sequence as set forth in SEQ ID NO: 5.

Functional antibody fragments comprising whole or essentially whole variable regions of both light and heavy chains are defined as follows:

(i) Fv, defined as a genetically engineered fragment consisting of the variable region of the light chain (VL) and the variable region of the heavy chain (VH) expressed as two chains;

(ii) single chain Fv (“scFv”), a genetically engineered single chain molecule including the variable region of the light chain and the variable region of the heavy chain, linked by a suitable polypeptide linker as a genetically fused single chain molecule.

(iii) disulfide-stabilized Fv (“dsFv”), a genetically engineered antibody including the variable region of the light chain and the variable region of the heavy chain, linked by a genetically engineered disulfide bond.

(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;

(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);

(vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds); and

(vii) Single domain antibodies or nanobodies are composed of a single VH or VL domains which exhibit sufficient affinity to the antigen.

According to specific embodiments the antibody heavy chain constant region is chosen from, e.g., IgG1, IgG2, IgG3, IgG4, IgM, IgA1, IgA2, IgD, and IgE.

According to specific embodiments, the antibody is an IgG antibody.

According to a specific embodiment the antibody isotype is IgG1 or IgG4.

According to a specific embodiment the antibody is IgG1 e.g. IgG1 kappa.

According to a specific embodiment the antibody is IgG2 e.g. IgG 2a, IgG2b e.g. IgG2a kappa or IgG2b kappa.

The choice of antibody type will depend on the immune effector function that the antibody is designed to elicit.

According to specific embodiments, the antibody comprises an Fc domain.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69: 2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242: 423-426 (1988); Pack et al., Bio/Technology 11: 1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

The antibody may be monospecific (capable of recognizing one epitope or protein), bispecific (capable of binding two epitopes or proteins) or multispecific (capable of recognizing multiple epitopes or proteins).

According to specific embodiments, the antibody is monospecific.

According to specific embodiments, the antibody is multispecific e.g. bispecific, trispecific, tetraspecific.

According to some embodiments of the invention, the antibody is bispecific.

Bispecific antibodies are artificial hybrid antibodies having two different heavy/light chain pairs and two different recognition (i.e. binding) sites that are capable of specifically binding at least two different epitopes. The different epitopes can either be within the same molecule or on different molecules such that the bispecific antibody can specifically recognize and bind two different epitopes on a single CD24 polypeptide as well as two different CD24 polypeptides. Alternatively, a bispecific antibody has a first recognition moiety having affinity to CD24 and a second recognition moiety having affinity for a polypeptide distinct from CD24, such as, but not limited to a polypeptide expressed by an immune cell.

Thus, according to specific embodiments, the multispecific antibody comprises an antigen recognition domain which specifically binds CD24 as disclosed herein and an antigen recognition domain which specifically binds an immune cell.

Non-limiting examples of immune cells include T cells, NK cells, NKT cells, B cells, macrophages, dendritic cells (DCs) and granulocytes.

According to specific embodiments, the immune cell is a T cell.

Non-limiting examples of polypeptides specifically expressed by immune cells include CD2, CD3, CD4, CD8, CD19, CD22, CD56, CD14, CD33, CD28, B7, CD64, CD32, CD16, PD1, CD68, CD11b.

According to specific embodiments, the polypeptide expressed by the immune cell is CD3.

Thus, according to specific embodiments, the multispecific (e.g. bispecific) antibody comprises an antigen recognition domain which specifically binds CD24 as disclosed herein and an antigen recognition domain which specifically binds CD3.

Numerous anti-CD3 antibodies are known in the art. Non-limiting examples of such anti-CD3 antibodies include OKT3, diL2K, TR66, UCHT1, humanized UHCT1, F6A.

According to specific embodiments, the antigen recognition domain which specifically binds CD3 comprises complementarity determining regions (CDRs) as set forth in SEQ ID NOs: 36, 37 and 39 arranged in a sequential order from N to C on a light chain of the antibody and CDRs as set forth in SEQ ID NOs: 44, 46 and 48 arranged in a sequential order from N to C on a heavy chain of said antibody.

According to specific embodiments, the VL of the antigen recognition domain which specifically binds CD3 is as set forth in SEQ ID NO: 34.

According to specific embodiments, the VH of the antigen recognition domain which specifically binds CD3 is as set forth in SEQ ID NO: 42.

According to a specific embodiment, the bispecific antibody comprises SEQ ID NO: 29 or 31.

According to a specific embodiment, the bispecific antibody is as set forth in SEQ ID NO: 29 or 31.

Methods of producing bispecific antibodies are known in the art and disclosed for examples in Songsivilai and Lachmann (1990) Clin. Exp. Immunol. 79: 315-321; Kostelny et al. (1992) J. Immunol. 148: 1547-1553, U.S. Pat. Nos. 4,474,893, 5,959,084 and 7,235,641, 7,183,076, US Publication Number 20080219980 and International Publication Numbers WO 2010/115589, WO2013150043 and WO2012118903 all incorporated herein by their entirety; and include, for example, chemical cross-linking (Brennan, et al., Science 229,81 (1985); Raso, et al., J. Biol. Chern. 272, 27623 (1997)), disulfide exchange, production of hybrid-hybridomas (quadromas), by transcription and translation to produce a single polypeptide chain embodying a bispecific antibody, or by transcription and translation to produce more than one polypeptide chain that can associate covalently to produce a bispecific antibody. The contemplated bispecific antibody can also be made entirely by chemical synthesis.

It will be appreciated that for human therapy or diagnostics, humanized antibodies are preferably used.

According to specific embodiments, the antibody is a humanized antibody. Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321: 522-525 (1986); Riechmann et al., Nature, 332: 323-329 (1988); and Presta, Curr. Op. Struct. Biol., 2: 593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321: 522-525 (1986); Riechmann et al., Nature 332: 323-327 (1988); Verhoeyen et al., Science, 239: 1534-1536 (1988)], by substituting rodent CDRs or CDR sequences for the corresponding sequences of a human antibody. Accordingly, such humanized antibodies are chimeric antibodies (U.S. Pat. No.

4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

According to specific embodiments, the antibody is a human antibody.

Human antibodies can also be produced using various techniques known in the art, including phage display libraries [Hoogenboom and Winter, J. Mol. Biol., 227: 381 (1991); Marks et al., J. Mol. Biol., 222: 581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1): 86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Once antibodies are obtained, they may be tested for activity, for example via ELISA.

According to specific embodiments, the antibody comprises a therapeutic moiety.

The therapeutic moiety can be proteinaceous or non-proteinaceous.

The Therapeutic moiety may be any molecule, including small molecule chemical compounds and polypeptides.

According to specific embodiments, the therapeutic moiety is capable of eliciting an immune response to a cell presenting CD24 on its cell surface.

As used herein, the phrase “eliciting an immune response” or “activating an immune cell” refers to stimulation of an immune cell (e.g. T cell, dendritic cell, macrophage, NK cell, B cell) that results in cellular proliferation, maturation, cytokine production, phagocytosis and/or induction of regulatory or effector functions.

Methods of evaluating immune cell activation or function are well known in the art and include, but are not limited to, proliferation assays such as BRDU and thymidine incorporation, cytotoxicity assays such as chromium release, cytokine secretion assays such as intracellular cytokine staining ELISPOT and ELISA, expression of activation markers such as CD25, CD69 and CD69 using flow cytometry and multimer (e.g. tetramer) assays, phagocytosis of target cells using flow cytometry.

The therapeutic moiety can be an integral part of the antibody e.g., in the case of a whole antibody, the Fc domain, which activates antibody-dependent cell-mediated cytotoxicity (ADCC). ADCC is a mechanism of cell-mediated immune defense whereby an effector cell of the immune system actively lyses a target cell, whose membrane-surface antigens have been bound by specific antibodies. It is one of the mechanisms through which antibodies, as part of the humoral immune response, can act to limit and contain infection. Classical ADCC is mediated by natural killer (NK) cells; macrophages, neutrophils and eosinophils can also mediate ADCC. For example, eosinophils can kill certain parasitic worms known as helminths through ADCC mediated by IgE. ADCC is part of the adaptive immune response due to its dependence on a prior antibody response.

Thus, according to specific embodiments, the therapeutic moiety is capable of eliciting antibody dependent cell toxicity (ADCC).

According to some embodiments of the invention, the therapeutic moiety is capable of eliciting complement-dependent cytotoxicity (CDC).

According to some embodiments of the invention, the therapeutic moiety is capable of eliciting antibody-dependent cellular phagocytosis (ADCP).

Alternatively or additionally, the antibody may be a bispecific antibody, as further described hereinabove, in which the therapeutic moiety is an immune cell engager such as an anti-CD3 antibody, an anti-CD16 or an anti-immune checkpoint molecule (e.g. anti PD-1).

Thus, according to an aspect of the present invention, there is provided a method of activating an immune cell towards a cell expressing CD24 in a subject in need thereof, the method comprising administering to the subject a therapeutically effective amount of the antibody, thereby activating the immune cell towards the cell expressing the CD24.

Alternatively or additionally, according to specific embodiments, the therapeutic moiety is an immune cell expressing the antibody. Non-limiting examples of immune cells that can be used with specific embodiments of the invention include T cells, NK cells, NKT cells, B cells, macrophages, dendritic cells (DCs) and granulocytes.

According to specific embodiments, the immune cell is a T cell.

Thus, according to specific embodiments, the antibody is part of a chimeric antigen receptor (CAR) and the therapeutic moiety is a T cell transduced with the agent.

A chimeric antigen receptor (CAR) is an artificially constructed hybrid protein or polypeptide containing an antigen binding domain of an antibody (e.g., a single chain variable fragment (scFv)) linked to T-cell signaling or T-cell activation domains. Method of generating CAR and transducing a T cell with a CAR are known in the art and are disclosed e.g. in Davila et al. Oncoimmunology. 2012 Dec 1; 1(9): 1577-1583; Wang and Rivière Cancer Gene Ther. 2015 Mar; 22(2): 85-94); Maus et al. Blood. 2014 Apr 24; (17): 2625-35; Porter DL The New England journal of medicine. 2011, 365(8): 725-733; Jackson H J, Nat Rev Clin Oncol. 2016; 13(6): 370-383; and Globerson-Levin et al. Mol Ther. 2014; 22(5): 1029-1038.

Alternatively or additionally the antibody may be attached to a heterologous therapeutic moiety (methods of conjugation are described hereinbelow). The therapeutic moiety can be, for example, a cytotoxic moiety, a toxic moiety [e.g., Pseudomonas exotoxin (GenBank Accession Nos. AAB25018 and S53109); PE38KDEL; Diphtheria toxin (GenBank Accession Nos. E00489 and E00489); Ricin A toxin (GenBank Accession Nos. 225988 and A23903)], a cytokine moiety [e.g., interleukin 2 (GenBank Accession Nos. CAA00227 and A02159), interleukin 10 (GenBank Accession Nos. P22301 and M57627)], a drug, a chemical, a protein and/or a radioisotope.

According to specific embodiments, the therapeutic moiety is selected from the group consisting of a toxin, a drug, a chemical, a protein and a radioisotope.

According to specific embodiments the antibody is bound to a detectable moiety. Examples of detectable moieties that can be used with some embodiments of the present invention include but are not limited to radioactive isotopes, phosphorescent chemicals, chemiluminescent chemicals, fluorescent chemicals, enzymes, fluorescent polypeptides, a radioactive isotope (such as ^{[125]}iodine) and epitope tags. The detectable moiety can be a member of a binding pair, which is identifiable via its interaction with an additional member of the binding pair, and a label which is directly visualized. In one example, the member of the binding pair is an antigen which is identified by a corresponding labeled antibody. In one example, the label is a fluorescent protein or an enzyme producing a colorimetric reaction.

Examples of suitable fluorophores include, but are not limited to, phycoerythrin (PE), fluorescein isothiocyanate (FITC), Cy-chrome, rhodamine, green fluorescent protein (GFP), blue fluorescent protein (BFP), Texas red, PE-Cy5, and the like. For additional guidance regarding fluorophore selection, methods of linking fluorophores to various types of molecules see Richard P. Haugland, “Molecular Probes: Handbook of Fluorescent Probes and Research Chemicals 1992-1994”, 5th ed., Molecular Probes, Inc. (1994); U.S. Pat. No. 6,037,137 to Oncoimmunin Inc.; Hermanson, “Bioconjugate Techniques”, Academic Press New York, N.Y. (1995); Kay M. et al., 1995. Biochemistry 34: 293; Stubbs et al., 1996. Biochemistry 35: 937; Gakamsky D. et al., “Evaluating Receptor Stoichiometry by Fluorescence Resonance Energy Transfer,” in “Receptors: A Practical Approach,” 2nd ed., Stanford C. and Horton R. (eds.), Oxford University Press, UK. (2001); U.S. Pat. No. 6,350,466 to Targesome, Inc.]. Fluorescence detection methods which can be used to detect the antibody when conjugated to a fluorescent detectable moiety include, for example, fluorescence activated flow cytometry (FACS), immunofluorescence confocal microscopy, fluorescence in-situ hybridization (FISH) and fluorescence resonance energy transfer (FRET).

Numerous types of enzymes may be attached to the antibody [e.g., horseradish peroxidase (HPR), beta-galactosidase, and alkaline phosphatase (AP)] and detection of enzyme-conjugated antibodies can be performed using ELISA (e.g., in solution), enzyme-linked immunohistochemical assay (e.g., in a fixed tissue), enzyme-linked chemiluminescence assay (e.g., in an electrophoretically separated protein mixture) or other methods known in the art [see e.g., Khatkhatay M I. and Desai M., 1999. J Immunoassay 20: 151-83; Wisdom GB., 1994. Methods Mol Biol. 32: 433-40; Ishikawa E. et al., 1983. J Immunoassay 4: 209-327; Oellerich M., 1980. J Clin Chem Clin Biochem. 18: 197-208; Schuurs A H. and van Weemen B K., 1980. J Immunoassay 1: 229-49).

Exemplary identifiable moieties include, but are not limited to green fluorescent protein, alkaline phosphatase, peroxidase, histidine tag, biotin, orange fluorescent protein and strepavidin.

Further examples of detectable moieties, include those detectable by Positron Emission Tomography (PET) and Magnetic Resonance Imaging (MRI), all of which are well known to those of skill in the art.

According to some embodiments, the therapeutic or detectable moieties are conjugated by translationally fusing the polynucleotide encoding the antibody disclosed herein with the nucleic acid sequence encoding the therapeutic or detectable moiety.

Additionally or alternatively, the therapeutic or detectable moieties can be chemically conjugated (coupled) to the antibody using any conjugation method known to one skilled in the art, including for example a 3-(2-pyridyldithio)propionic acid Nhydroxysuccinimide ester (also called N-succinimidyl 3-(2pyridyldithio) propionate) (“SDPD”) (Sigma, Cat. No. P-3415; see e.g., Cumber et al. 1985, Methods of Enzymology 112: 207-224), a glutaraldehyde conjugation procedure (see e.g., G. T. Hermanson 1996, “Antibody Modification and Conjugation, in Bioconjugate Techniques, Academic Press, San Diego) or a carbodiimide conjugation procedure [see e.g., J. March, Advanced Organic Chemistry: Reaction's, Mechanism, and Structure, pp. 349-50 & 372-74 (3d ed.), 1985; B. Neises et al. 1978, Angew Chem., Int. Ed. Engl. 17: 522; A. Hassner et al. 1978, Tetrahedron Lett. 4475; E. P. Boden et al. 1986, J. Org. Chem. 50: 2394 and L. J. Mathias 1979, Synthesis 561].

A therapeutic or detectable moiety can be attached, for example, to the antibody of some embodiments of the invention using standard chemical synthesis techniques widely practiced in the art [see e.g., hypertexttransferprotocol://worldwideweb (dot) chemistry (dot) org/portal/Chemistry)], such as using any suitable chemical linkage, direct or indirect, as via a peptide bond (when the functional moiety is a polypeptide), or via covalent bonding to an intervening linker element, such as a linker peptide or other chemical moiety, such as an organic polymer. Chimeric peptides may be linked via bonding at the carboxy (C) or amino (N) termini of the peptides, or via bonding to internal chemical groups such as straight, branched or cyclic side chains, internal carbon or nitrogen atoms, and the like. Description of fluorescent labeling of antibodies is provided in details in U.S. Pat. Nos. 3,940,475, 4,289,747, and 4,376,110.

The antibody can also be attached to particles which comprise the therapeutic or detectable moiety (e.g. cytotoxic agent). Methods of covalently binding an antibody to an encapsulating particle are known in the art and disclosed for example in U.S. Pat. Nos. 5,171,578, 5,204,096 and 5,258,499.

According to a specific embodiment, the antibody is generated using recombinant DNA techniques.

Hence, according to an aspect of the present invention there is provided a polynucleotide encoding the antibody. Such a polynucleotide will comprise the nucleic acid sequences encoding the CDRs.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

Non-limiting Examples of such nucleic acid sequences are provided in SEQ ID NOs: 49, 53, 30 or 32.

Host cells comprising the polynucleotide encoding the antibody are also contemplated herein. Thus, according to an aspect of the present invention, there is provided host cell expressing the antibody.

Such cells are typically selected for high expression of recombinant proteins (e.g., bacterial, plant or eukaryotic cells e.g., CHO, HEK-293 cells), but may also be an immune cell (e.g., macrophages, dendritic cells, T cells, B cells or NK cells) when for instance the CDRs of the antibody are implanted in a CAR transduced in said cells which are used in adoptive cell therapy.

To express any of the disclosed antibodies in a cell, a polynucleotide sequence encoding the antibody is preferably ligated into a nucleic acid construct suitable for cell expression and introduced into the host cell. Such a nucleic acid construct includes a promoter sequence for directing transcription of the polynucleotide sequence in the cell in a constitutive or inducible manner.

The nucleic acid construct (also referred to herein as an “expression vector”) of some embodiments of the invention includes additional sequences which render this vector suitable for replication and integration (e.g., shuttle vectors). In addition, a typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

It will be appreciated that the individual elements comprised in the expression vector can be arranged in a variety of configurations. For example, enhancer elements, promoters and the like, and even the polynucleotide sequence(s) encoding the polypeptide can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p205. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Various methods can be used to introduce the expression vector of some embodiments of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

According to an additional or an alternative aspect of the present invention, there is provided a method of producing an anti-CD24 antibody, the method comprising:

(a) culturing a host cell expressing the antibody under conditions which support production of said antibody; and

(b) recovering said antibody.

Such conditions may be for example an appropriate temperature (e.g., 37° C.), atmosphere (e.g., air plus 5% CO2), pH, light, medium, supplements and the like.

According to a specific embodiment, the antibody is isolated (purified) from the culture. According to specific embodiments, the isolated antibody is essentially free from contaminating cellular components such as carbohydrate, lipid or other impurities.

Methods for isolation and purification of antibodies are well known in the art, see for example Chromatography, 5^{th} edition, Part A: Fundamentals and Techniques, Heftmann, E. (ed), Elsevier Science Publishing Company, New York, (1992); Advanced Chromatographic and Electromigration Methods in Biosciences, Deyl, Z. (ed.), Elsevier Science B V, Amsterdam, The Netherlands, (1998); Chromatography Today, Poole, C. F., and Poole, S. K., Elsevier Science Publishing Company, New York, (1991); Scopes, Protein Purification: Principles and Practice (1982); Sambrook, J., et al. (ed), Molecular Cloning: A Laboratory Manual, Second Edition, Cold Spring Harbor Laboratory Press, Cold Spring Harbor, N.Y., 1989; or Current Protocols in Molecular Biology, Ausubel, F. M., et al. (eds), John Wiley & Sons, Inc., New York.

According to specific embodiments, at least 80%, at least 90%, at least 95% or at least 99% of the total protein in the preparation is the antibody of interest.

According to specific embodiments, the isolated antibody is purified to a pharmaceutically acceptable purity.

Methods for evaluating purity are well known in the art and include SEC-HPLC, peptide mapping, SDS gel analysis and ELISA for specific contaminants.

The antibodies disclosed herein can be used in a variety of clinical applications. By virtue of their affinity to CD24 they can be used in the treatment of CD24 associated medical conditions such as cancer.

Thus, according to an aspect of the present invention, there is provided a method of treating a disease associated with cells expressing CD24 in a subject in need thereof, the method comprising administering to the subject a therapeutically effective amount of the antibody, thereby treating the disease in the subject.

According to an additional or an alternative aspect of the present invention, there is provided the antibody, for use in treating a disease associated with cells expressing CD24 in a subject in need thereof.

As used herein, the term “subject” includes mammals, preferably human beings at any age or gender which suffer from the pathology. Preferably, this term encompasses individuals who are at risk to develop the pathology.

As used herein the phrase, “disease associated with cells expressing CD24” means that cells expressing CD24 drive onset and/or progression of the disease.

According to specific embodiments, the cells associated with the disease overexpress CD24.

According to specific embodiments, the expression of CD24 on the cells is at least 2%, at least 5%, at least 10%, at least 20%, at least 30%, at least 40%, at least 50%, at least 60%, at least 70%, at least 80%, at least 90%, at least 100% or higher as compared the level of CD24 on healthy cells, as determined by e.g. flow cytometry.

Non-limiting examples such disease include cancer, inflammatory bowel disease (e.g. UC and Crohn), nephrological disorder [e.g. Acute tubular necrosis (ATN)], cardiovascular disease (e.g. myocardial infarction), Eosinophilic esophagitis (EOE), pulmonary disease (e.g. asthma).

Cancers which may be treated by some embodiments of the invention can be any solid or non-solid tumor, cancer metastasis and/or a pre-cancer.

Examples of cancer include but are not limited to, carcinoma, blastoma, sarcoma and lymphoma. More particular examples of such cancers include, but are not limited to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic ; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute—megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

Precancers are well characterized and known in the art (refer, for example, to Berman J J. and Henson D E., 2003. Classifying the precancers: a metadata approach. BMC Med Inform Decis Mak. 3: 8). Examples of precancers include but are not limited to include acquired small precancers, acquired large lesions with nuclear atypia, precursor lesions occurring with inherited hyperplastic syndromes that progress to cancer, and acquired diffuse hyperplasias and diffuse metaplasias. Non-limiting examples of small precancers include HGSIL (High grade squamous intraepithelial lesion of uterine cervix), AIN (anal intraepithelial neoplasia), dysplasia of vocal cord, aberrant crypts (of colon), PIN (prostatic intraepithelial neoplasia).

Non-limiting examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma. Non-limiting examples of precursor lesions occurring with inherited hyperplastic syndromes that progress to cancer include atypical mole syndrome, C cell adenomatosis and MEA. Non-limiting examples of acquired diffuse hyperplasias and diffuse metaplasias include Paget's disease of bone and ulcerative colitis.

According to specific embodiments, the cancer is selected from the group consisting of colorectal cancer, pancreatic cancer, B-cell lymphomas, glioma, small-cell lung cancer, non-small cell lung, hepatic cancer, renal cancer, nasopharyngeal cancer, bladder cancer, uterine cancer, ovarian cancer, breast cancer and prostate cancer.

According to specific embodiments, the cancer is colorectal cancer or pancreatic cancer.

The antibody of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the anti-CD24 antibody accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to specific embodiments, the antibody can be administered to a subject in combination with other established or experimental therapeutic regimen to treat a disease associated with cells expressing CD24 (e.g. cancer) including, but not limited to analgesics, chemotherapeutic agents, radiotherapeutic agents, phototherapy and photodynamic therapy, cytotoxic therapies (conditioning), hormonal therapy, immunotherapy, cellular therapy, and other treatment regimens (e.g., surgery) which are well known in the art.

According to specific embodiments, the therapy is selected from the group consisting of immunotherapy, chemotherapy and radiotherapy.

Thus, according to specific embodiments, the method further comprises administering to the subject a therapy for treating the disease.

According to an aspect of the present invention there is provided an article of manufacture comprising a packaging material packaging the antibody disclosed herein; and a therapy for treating a disease associated with cells expressing CD24.

According to specific embodiments, the article of manufacture is identified for the treatment of a disease associated with cells expressing CD24 (e.g. cancer).

According to specific embodiments, the antibody and the therapy are packaged in separate containers.

According to specific embodiments, the antibody and the therapy are packaged in a co-formulation.

Non-limiting examples of anti-cancer agent that can be used with specific embodiments of the invention include, but are not limited to the anti-cancer drugs Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta- I a; Interferon Gamma- I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

According to specific embodiments, the anti-cancer agent comprises an antibody.

Non-limiting examples of such antibodies include rituximab, cetuximab, trastuzumab, edrecolomab, alemtuzumab, gemtuzumab, ibritumomab, panitumumab Belimumab, Bevacizumab, Bivatuzumab mertansine, Blinatumomab, Blontuvetmab, Brentuximab vedotin, Catumaxomab, Cixutumumab, Daclizumab, Adalimumab, Bezlotoxumab, Certolizumab pegol, Citatuzumab bogatox, Daratumumab, Dinutuximab, Elotuzumab, Ertumaxomab, Etaracizumab, Gemtuzumab ozogamicin, Girentuximab, Necitumumab, Obinutuzumab, Ofatumumab, Pertuzumab, Ramucirumab, Siltuximab, Tositumomab, Nivolumab, Pembrolizumab, Durvalumab, Atezolizumab, Avelumab Trastuzumab and ipilimumab.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition (pathology, e.g. cancer) or substantially preventing the appearance of clinical or aesthetical symptoms of a condition (pathology, e.g. cancer). Those of skill in the art will understand that various methodologies and assays can be used to assess the development of a pathology, and similarly, various methodologies and assays may be used to assess the reduction, remission or regression of a pathology (e.g. cancer).

According to specific embodiments, treatment may be evaluated by a decrease in tumor volume, a decrease in the number of tumor cells, a decrease in the number of metastases, an increase in life expectancy, or amelioration of various physiological symptoms associated with the cancerous condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells - A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Genetically Engineering A Humanized Anti-CD24 Monoclonal Antibody

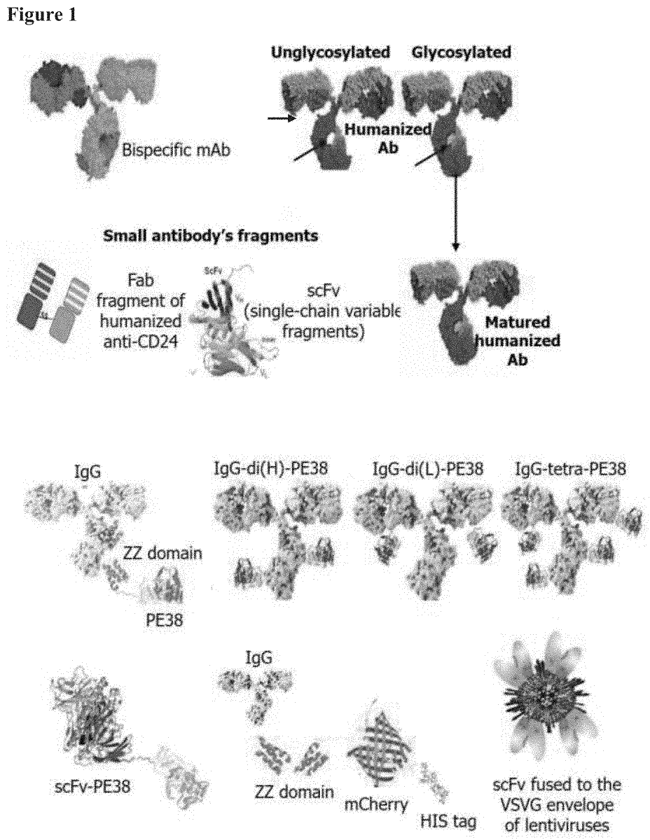

The present inventors have constructed several derivatives of the humanized anti-CD24 murine SWA11 mAb (Table 1 hereinbelow). Following, several antibodies were developed by means of genetic engineering, ranging from big (whole IgG) to small (scFv and Fab), including un-armed and conjugated derivatives (FIG. 1).

A bioinformatic approach was undertaken to select the best human variable domains that should serve as framework donors. In general, humanization is based on using the human sequence with maximal similarity. However, additional criteria such as the ability of the selected human sequence to support the canonical structures of the murine antibody CDR loops had been considered as well.

First, the sequences of the VH and VK RT-PCR products that were synthesized from cellular RNA prepared from anti-CD24 SWA11 hybridoma cells were aligned with the database of mouse immunoglobulin germline gene sequences using IgBlast. It was found that SWA11 VH was derived from the VH36-60a1.85 germline gene (GenBank accession AJ851868) while SWA11 VK was derived from the 8-30 germline gene (GenBank accession AJ235948).

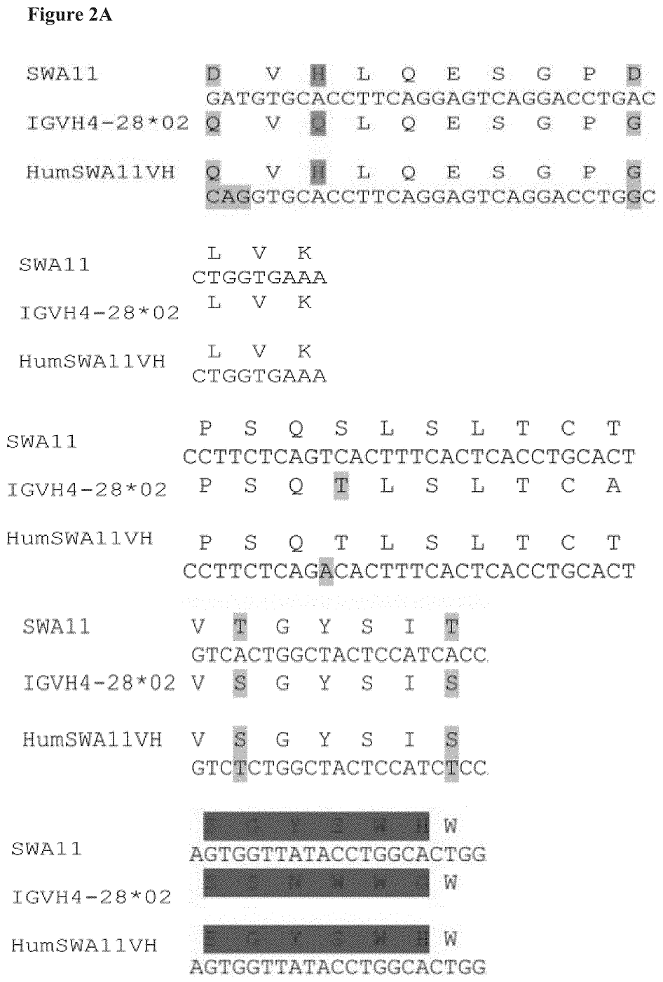

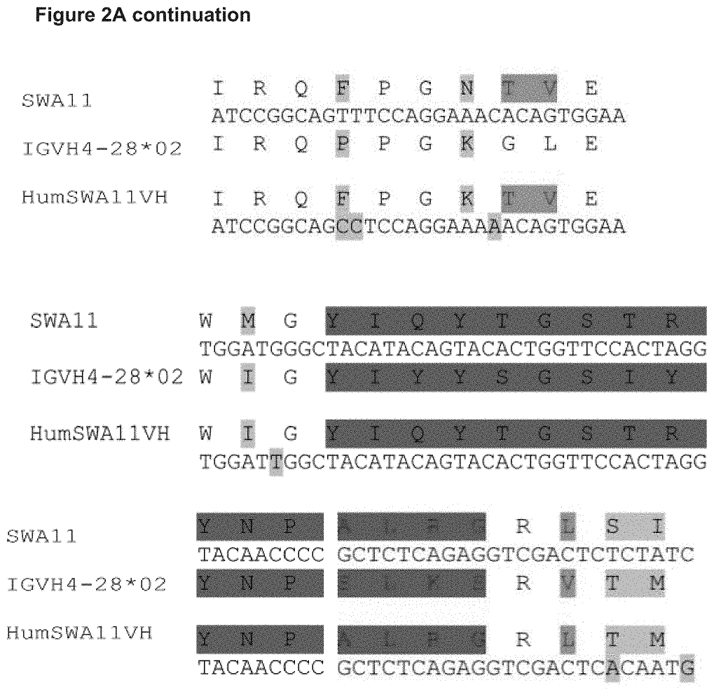

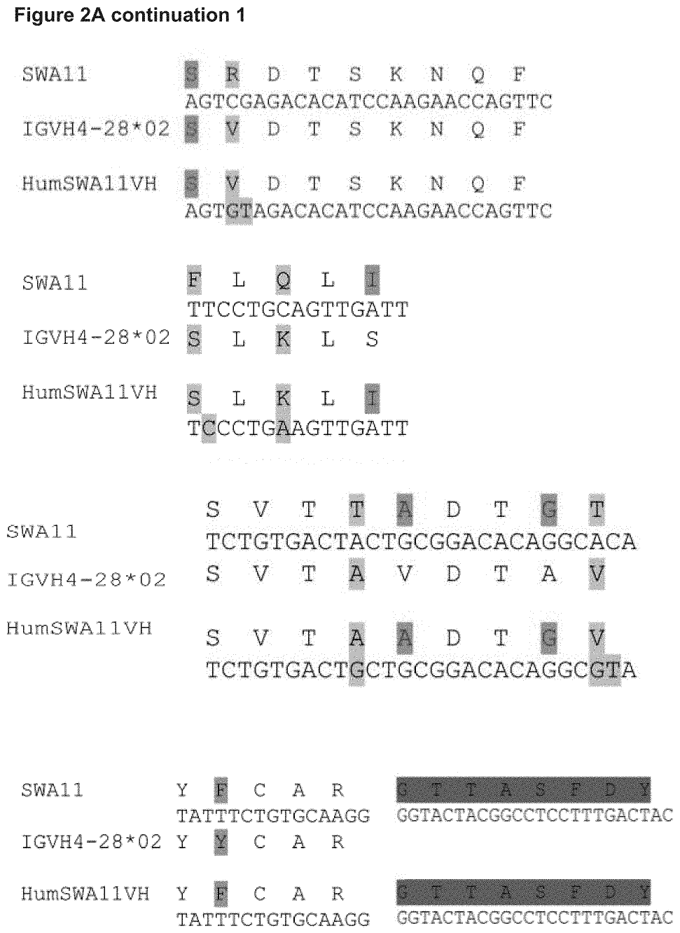

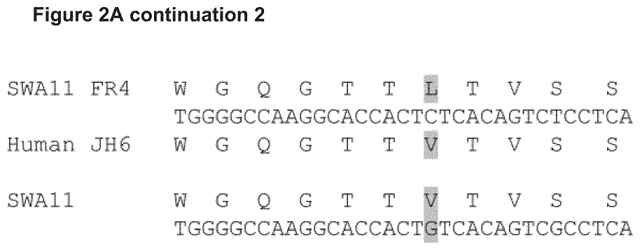

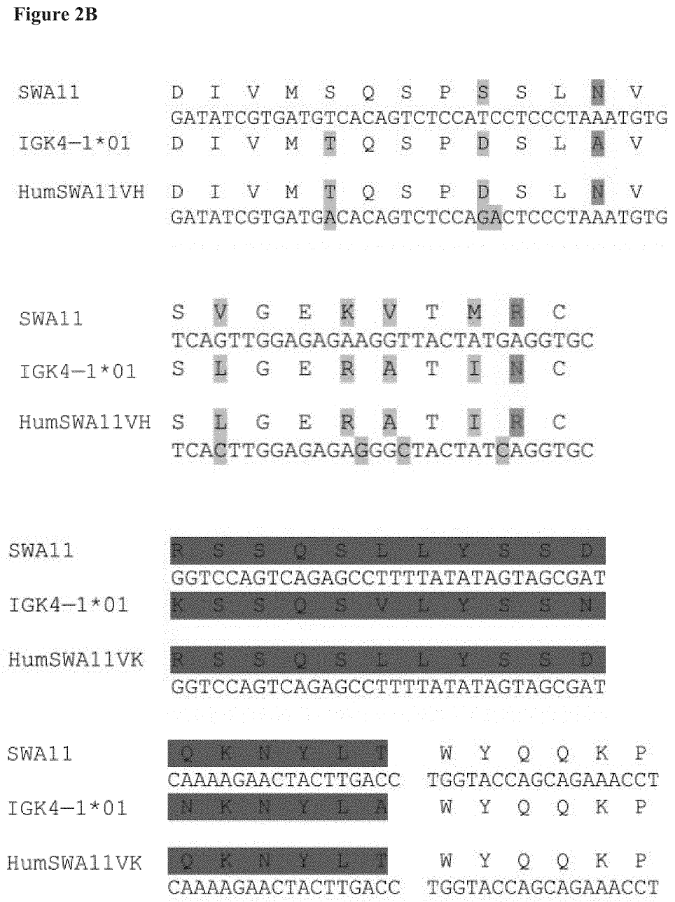

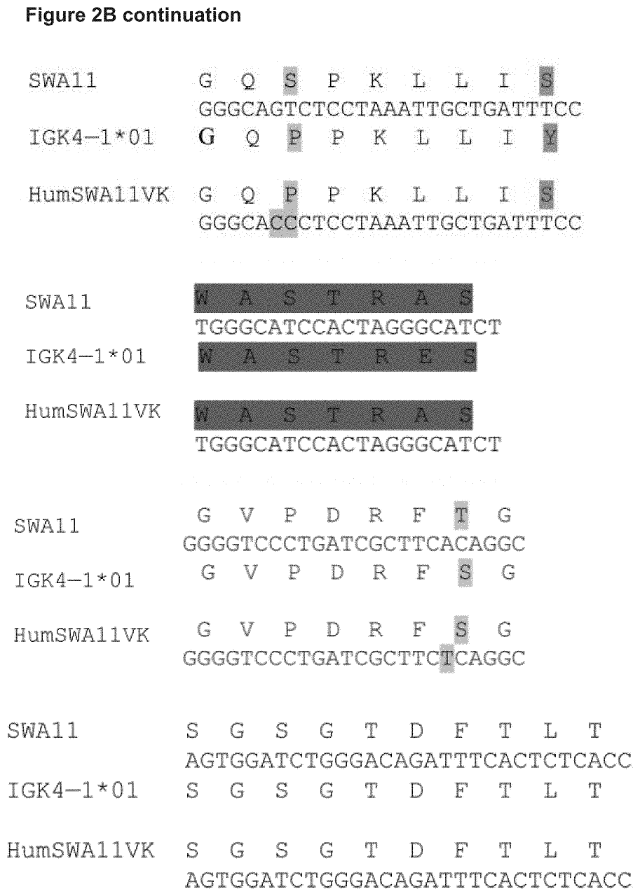

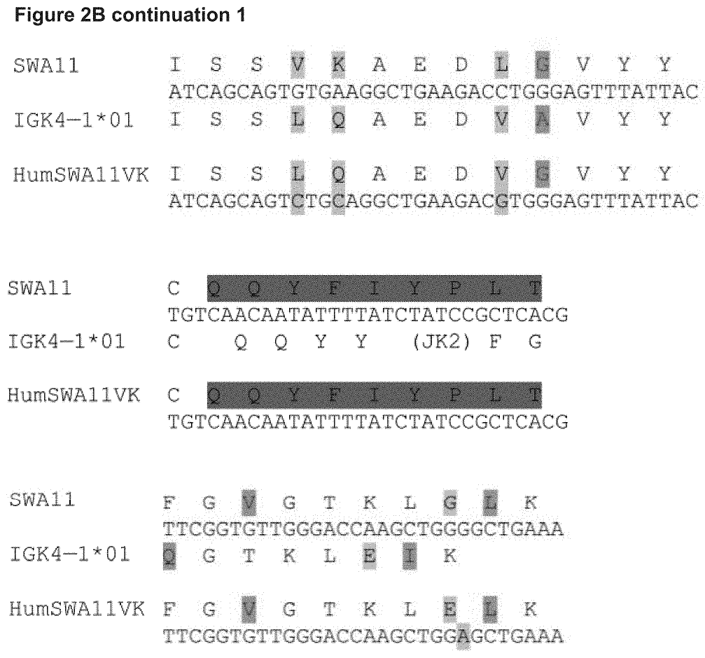

Following, to select suitable human variable domains that should serve as framework donors, the amino acid sequences of VH36-60a1.85 and 8-30 were independently aligned against the entire repertoire of human antibody sequences contained in the GenBank database using Igbalst. The comparison with human immunoglobulin genes revealed that the SWA11 VH gene had a high level of identity with the deduced amino acid sequence of the human VHIV family germline V gene 28*02 (GenBank accession M83133). The alignment of SWA11 VH with IGVH-28*02 is shown in FIG. 2A. SWA11 VK gene showed a high level of sequence identity with the deduced amino acid sequence of the human IGK4-1*01 (GenBank accession AF017732). The alignment of SWA11 VK with the deduced amino acid sequence of the human IGK4 is shown in FIG. 2B.

Following, the replacements found between SWA11 to 28*02 and 1*01 were introduced at the DNA level. In addition, for cloning into expression vectors, the VH and VK genes were synthesized with the addition of appropriate restriction sites.

TABLE 1
description of the constructed humanized anti-CD24 antibody derivatives.

| SEQ ID NO | Plasmid | Encoded polypeptide | Description |
|---|---|---|---|
| 9 | pMAZ- | Chimeric heavy chain | Plasmid carrying the murine SWA11 V_{H} |
| | IgH(chSWA11) | of anti-CD24 mAb | and human CH1-CH3 |
| 10 | pMAZ- | Chimeric light chain of | Plasmid carrying the murine SWA11 V_{L} and |
| | IgL(chSWA11) | anti-CD24 mAb | human Cₖ. |
| 11 | pMAZ- | Humanized heavy | Plasmid carrying the humanized anti-CD24 IgH |
| | IgH(HuSWA11) | chain of anti-CD24 | |
| | | mAb | |
| 12 | pMAZ- | Humanized light chain | Plasmid carrying the humanized IgL |
| | IgL(HuSWA11) | of anti-CD24 mAb | |
| 13 | pcDNA4-TetO₂- | Humanized light and | An eukaryotic tetracycline-regulated expression |
| | CMV-IgL-TetO₂- | heavy chains of anti- | vector encoding for humanized heavy and light |
| | CMV-IgH | CD24 mAb | chains of anti-CD24 mAb. The gene encoding |
| | | | for zeocin was replaced with the heavy chain |
| | | | gene, and the SV40 promoter was replaced with |
| | | | pCMV-TetO₂. |
| 11 | pHAK- | Humanized heavy | Plasmid carrying the humanized anti-CD24 IgH |
| | humanized-IgH | chain of anti-CD24 | for expression in E.coli as insoluble protein |
| | | mAb | |
| 12 | pHAK- | Humanized light chain | Plasmid carrying the humanized anti-CD24 IgL |
| | humanized-IgL | of anti-CD24 mAb | for expression in E.coli as insoluble protein |
| 14 | pET28a-HIS-Hu- | Humanized light chain | Plasmid carrying the humanized anti-CD24 IgL. |
| | IgL | of anti-CD24 mAb | HIS-tag is fused at the N-terminus of the |
| | | | variable region. |

In the next step, large-scale production of the designed humanized anti-CD24 antibody referred to herein as HuNS17 was performed in order to evaluate its stability and activity in vitro and in vivo.

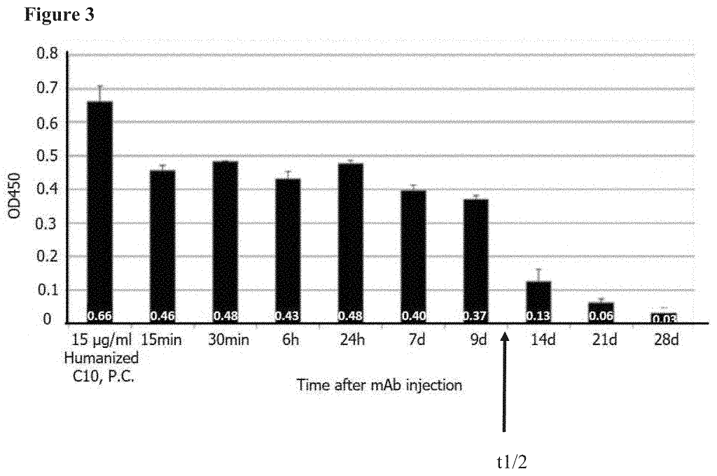

Stability pharmacokinetic studies showed that the antibody was stable (FIG. 3 and Table 2 hereinbelow). Specifically, the t1/2 of the humanized Ab was 5.2 days, the mean residence time was 9.2 days and the tmax was 60 minutes.

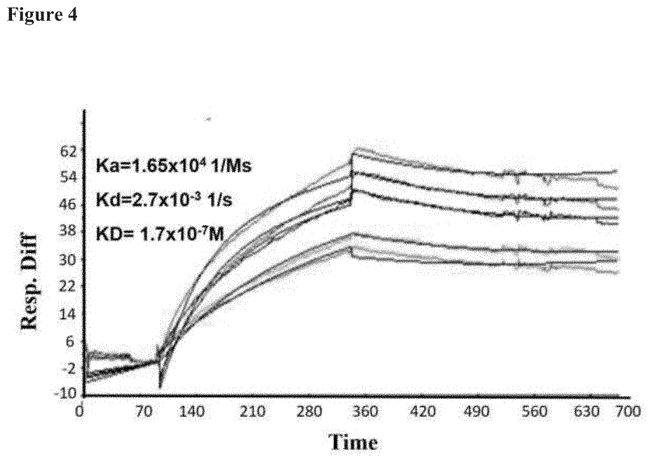

The ability of the generated antibody to specifically bind CD24 and to inhibit cell proliferation was tested by several bioassays and in various cell lines, specifically human colorectal cancer and pancreatic cancer cell lines. The results demonstrated that the purified humanized Ab was functional and had high binding strength and selectivity towards CD24. Specifically, the binding strength (e.g. affinity) of the anti-CD24 derivative was analyzed using Biacore analysis (Weizmann Institute of Science) (FIG. 4). The murine parental antibody was compared to the chimeric and humanized antibody forms. The different clones and batches of the Abs were also compared. In these evaluations the CD24 antigen was captured on the surface of the sensor chip and the different antibodies were the analytes. The results indicated that the differences between the humanized and the murine Abs are due to the Kd constant. The dissociation of the humanized Ab was faster than the murine one. However, the analysis was repeated by capturing the antibody on the chip, while the CD24 antigen was the flowing analyte. In this case, the Kd of the humanized Ab was 1.7×10⁻⁷ (while the murine was 3.27×10⁻⁸ and the chimeric mAb 1.5×10⁻⁷).

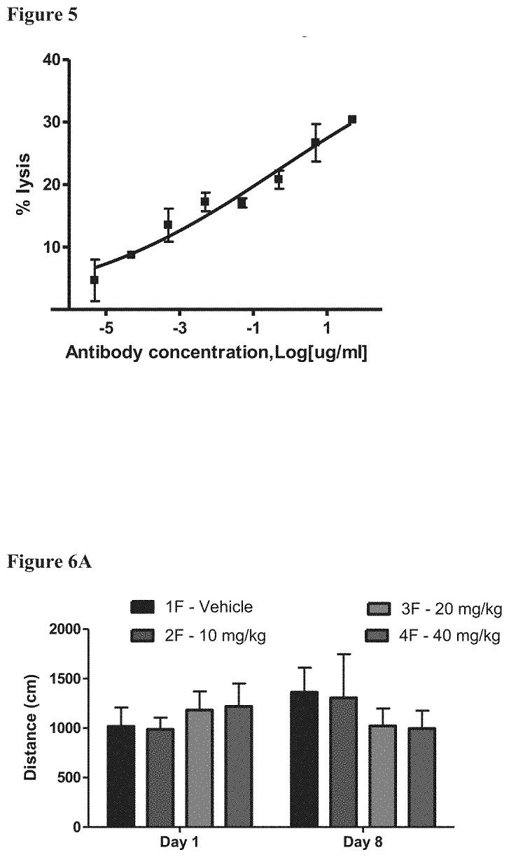

In addition, the anti-CD24 humanized mAb induced cell death by antibody-dependent cellular cytotoxicity (ADCC) (FIG. 5).

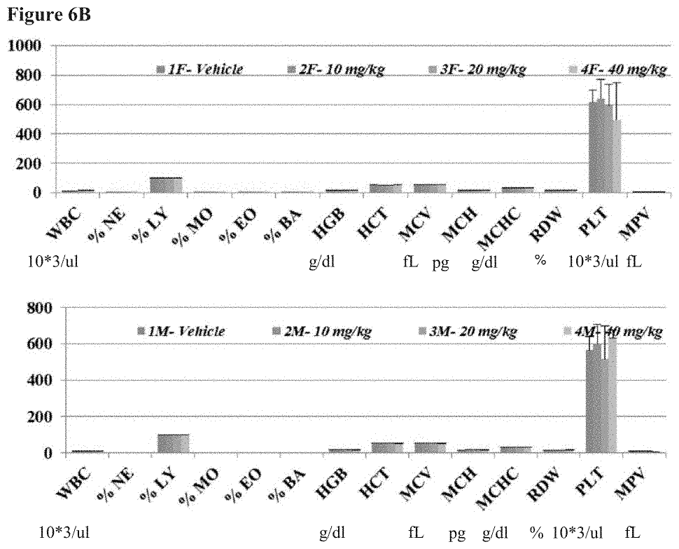

Moreover, acute intravenous maximum tolerated dose (MTD) studies using the humanized antibody in order to discover the highest tolerated dose without causing overt toxicity, and estimate an approximate concentration range for the humanized Ab for animals were effected (Table 3 hereinbelow and FIGS. 6A-B). No mortalities were recorded and significant clinical signs such as, lethargy, hypothermia, vasodilation or vasoconstriction, neuromuscular effects, were not observed. In addition, no differences in complete blood count or pathological examination were shown.

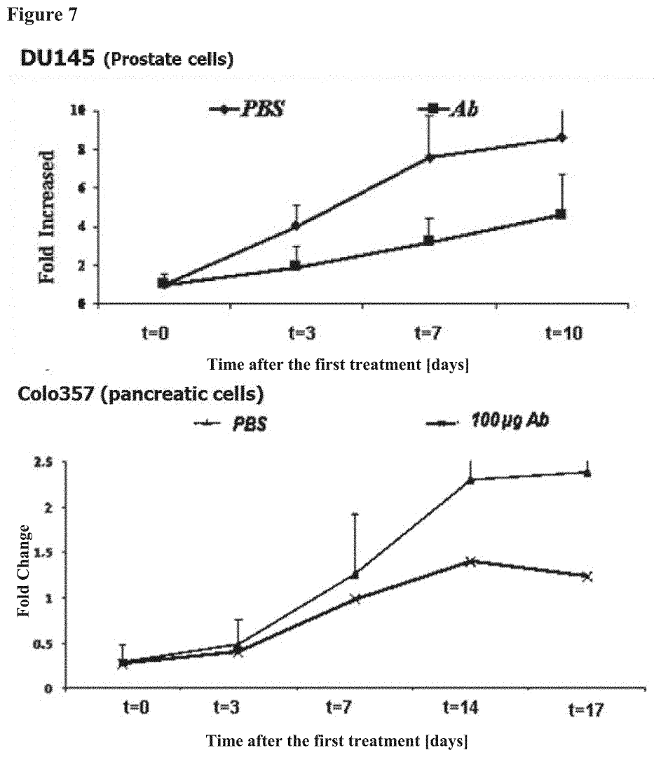

Following, the therapeutic effect and efficacy of anti-CD24 Abs in vivo was evaluated in a xenograft model of pancreatic and prostate cancers. The results, as demonstrated in FIG. 7, show that the humanized antibody inhibited tumor development and growth.

Taken together, the genetic manipulation during the humanization process did not harm the antibodies functional characteristics.

TABLE 2
Results of the pharmacokinetic (PK) studies

| | | # of | | Time | Time | Half- | | | | |
|---|---|---|---|---|---|---|---|---|---|---|
| Mouse | Rsq | point | Lambda z | lower | upper | life (hr) | tmax | cmax | AUC | MRT |
| 1 | 0.9647 | 4 | 0.0047 | 168 | 504 | 147.6 | 0.25 | 0.46 | 124.7 | 223.0 |
| 2 | 0.7677 | 4 | 0.0064 | 168 | 504 | 108.0 | 0.25 | 0.49 | 107.8 | 164.5 |
| 3 | 0.9511 | 6 | 0.0043 | 168 | 672 | 159.9 | 0.25 | 0.48 | 106.4 | 230.3 |
| 4 | 0.889 | 4 | 0.0074 | 168 | 456 | 93.6 | 0.25 | 0.36 | 104.8 | 181.7 |
| 5 | 0.9879 | 4 | 0.0062 | 216 | 672 | 112.5 | 0.5 | 0.48 | 148.7 | 197.3 |
| 6 | 0.8809 | 4 | 0.0125 | 168 | 504 | 55.6 | 0.25 | 0.49 | 85.6 | 110.8 |
| 8 | 0.9999 | 4 | 0.0093 | 168 | 456 | 74.3 | 0.5 | 0.4 | 88.6 | 148.0 |
| 10 | 0.9824 | 3 | 0.0041 | 72 | 336 | 167.4 | 0.25 | 0.48 | 176.3 | 397.1 |
| 11 | 0.8897 | 4 | 0.0035 | 24 | 216 | 198.5 | 1 | 0.47 | 142.6 | 335.3 |

TABLE 3
Study design for acute intravenous maximum tolerated
dose (MTD) evaluation.

| | | | Dose | Dose | |
|---|---|---|---|---|---|
| | Number | | Level | Volume | Route of |
| Group No. | of mice | Treatment | (mg/kg) | (ml/kg) | administration |
| 1M | 3 | Vwhicle | NA | 10.0 | IV |
| 1F | 3 | | | | |
| 2M | 3 | Anti-CD24 | 10 | | |
| 2F | 3 | mAb | | | |
| 3M | 3 | | 20 | | |
| 3F | 3 | | | | |
| 4M | 3 | | 40 | | |
| 5F | 3 | | | | |

### Example 2

### In-Vitro Phage Display Affinity Maturation of the Humanized Anti-CD24 Monoclonal Antibody

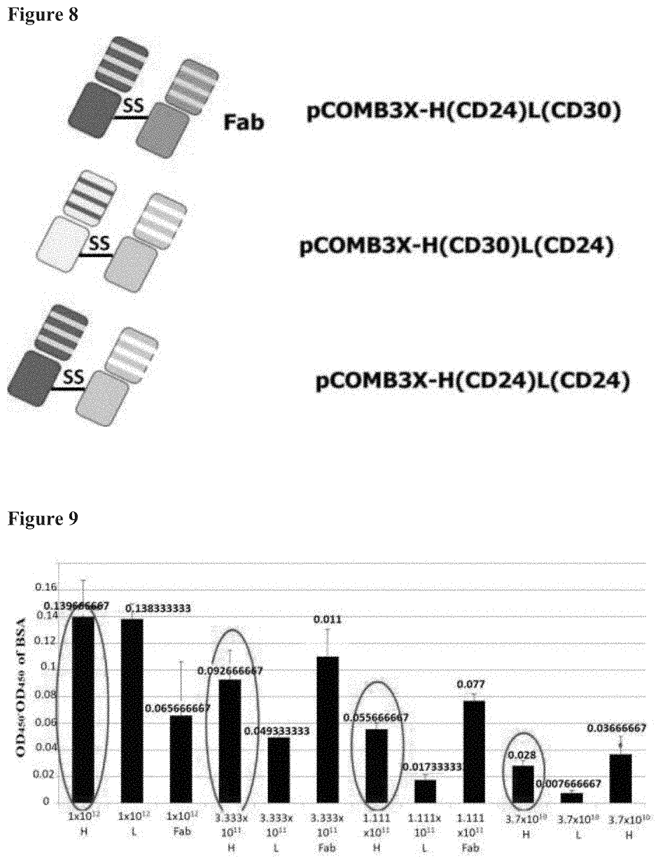

To understand which of the heavy and light chain contributes more to the binding pocket of anti-CD24 antibody a phage display technique was used. To this end, three plasmids were constructed which encode for the Fab fragment of anti-CD24 and/or anti-CD30, as described hereinbelow. The first construct contained both heavy and light chains of the humanized anti-CD24 humanized Ab; the second construct contained the heavy chain of anti-CD30 Ab and the light chain of the humanized anti-CD24 Ab; and the third contained the heavy chain of the humanized anti-CD24 Ab and the light chain of the anti-CD30 Ab (FIG. 8).

Construction of pCOMB3X-H(CD24)L(CD24)plasmid—The humanized VH domain was amplified from the pcDNA4-PCMV-TetO2-IgL-PCMV-TetO2-IgH (see Table 1 hereinabove) using primers HumSWA11-Fd-NcoI-FOR and HumSWA11-Fd-STOP-BspEI-FOR (see Table 4 hereinbelow) and introduced into the pCOMB3X vector as a NcoI/BspEI fragment, the resulting vector was named pCOMB3X-H(CD24). The humanized VL domain was amplified from the pcDNA4-PCMV-TetO2-IgL-PCMV-TetO2-IgH (see Table 1 hereinabove) using primers HumSWA11-L-SfiI-FOR and HumSWA11-L-STOP-XbaI-REV (see Table 4 hereinbelow) and introduced into the pCOMB3X-H(CD24) intermediate vector as a SfiI/XbaI fragment, the resulting vector was named pCOMB3X-H(CD24)L(CD24) (SEQ ID NO: 15).

Construction of pCOMB3X-H(CD30)L(CD24)plasmid—The pCOMB3X-H(CD30)L(CD24) plasmid was generated by digestion of pCOMB3X-H(CD24)L(CD24) and pCOMB3X-H(CD30)L(CD30) (Haim et al., mAb, 2009; Lilah et al., antibodies, 2018) plasmids by NcoI and BspEI and cloning of the second cleaved product into the first; the resulting vector was named pCOMB3X-H(CD30)L(CD24) (SEQ ID NO: 16).

Construction of pCOMB3X-H(CD24)L(CD30)plasmid—The pCOMB3X-H(CD24)L(CD30) plasmid was generated by digestion of pCOMB3X-H(CD24)L(CD24) and pCOMB3X-H(CD30)L(CD30) plasmids by Sfil and Xbal and cloning of the second cleaved product into the first; the resulting vector was named pCOMB3X-H(CD24)L(CD30) (SEQ ID NO: 17).

TABLE 4
list of primers

| | | SEQ |
|---|---|---|
| | | ID |
| Name | Sequence | NO: |
| HumSWA11-L- | gctaccgtggcccag | 18 |
| SfiI-FOR | gcggccGATATCGTG | |
| | ATGACACAGTCTCC | |
| HumSWA11-L- | attaattaTCTAGAt | 19 |
| STOP-XbaI- | taTTAACACTCTCCC | |
| REV | CTGTTGAAGC | |
| HumSWA11-Fd- | ccaaccagCCATGGc | 20 |
| NcoI-FOR | cCAGGTGCACCTTCA | |
| | GGAGTCAGG | |
| HumSWA11-Fd- | aagcgtagTCCGGAC | 21 |
| STOP-BspFI- | TAGTTTTGTCACAAG | |
| FOR | ATTTGGGCTCAACTC | |
| | TC | |
| 1 | cgcgattgcagtggc | 22 |
| | actgg | |
| 2 | GGAAGATGAAGACaG | 23 |
| | ATGGTGC | |
| 3 | CTATCAGGTGCAGGT | 24 |
| | CCAGTCMGASCSTTY | |
| | TCTATARCRGCGMCC | |
| | AAARATGGTACCAGC | |
| | AGAAACCT | |
| 4 | ACTGGACCTGCACCT | 25 |
| | GATAG | |
| 5 | CCTCCTAAATTGCTG | 26 |
| | ATTkgggactycmct | |
| | agggmaGGGGTCCCT | |
| | GATCGCTTC | |
| 6 | AATCAGCAATTTAGG | 27 |
| | AGG | |

Following, each Fab fragment was displayed on the surface of phages by fusing each Fab to the pIII protein of the M13 bacteriophage, each fusion protein was displayed as a single copy. The filamentous phages infect F+ E. coli bacteria via the sex pili. The procedure was performed as described in Benhar et al., (current Protocols in Immunology, 2002). Briefly, an exponential culture of E. coli TG-1 cells was prepared in YTAG medium (2YT medium complemented with 1% glucose and 100 μg/ml ampicillin). The cells were infected by adding 10⁹ PFU of helper phage (M13K07) to the culture in order to rescue the phagemids. On the following day, phagemids were concentrated and any soluble antibodies were removed by precipitating with PEG/NaCl). Following, the rescued phages were recovered and the titer of the phages was determined by live count and was confirmed by OD measuring. Binding of the three Fab derivatives to CD24 was examined by antigen-based ELISA (phage ELISA) (FIG. 9).

As expected, the highest signal was obtained with pCOMB3X-H(CD24)L(CD24) which contains the two anti-CD24 chains. However, comparing the signals obtained with pCOMB3X-H(CD30)L(CD24) and pCOMB3X-H(CD24)L(CD30) indicated that the heavy chain of the anti-CD24 Ab contributes more to the binding pocket. This result indicated higher chances of improving the anti-CD24 binding strength by mutagenesis of the light chain.

Therefore, in the next step affinity maturation procedure was effected on the humanized anti-CD24 light chain.

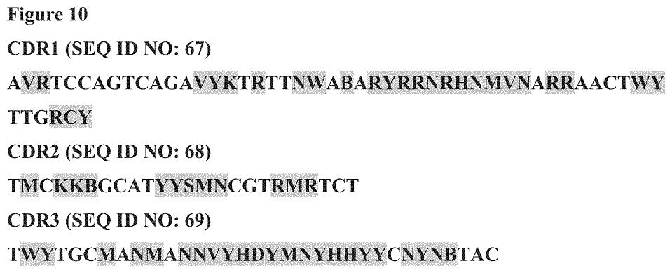

To this end, sequence analysis using the IgBlast was conducted in order to find the light chain CDR diversity. Bioinformatics analysis was performed and the Abs generated were compared to other 100 matured mAbs in order to find the specific residues in the CDR loops that were changed, i.e. CDR diversity. The search was performed on 100 homologues. As the size of the libraries is limited, amino acid sites that were changed only in one or two homologues were ignored. Three libraries were designed, one for each CDR (FIG. 10). Accordingly, the diversity of each CDR was calculated (Table 5 hereinbelow).

TABLE 5
Diversity of each CDR

| CDR | CDR Length (aa) | Diversity |
|---|---|---|
| 1 | 17 | 5.8 × 10⁵ |
| 2 | 8 | 180 |
| 3 | 11 | 4.18 × 10⁶ |

The designed libraries where sent to GeneArt® (Life Technologies) for synthesis. Of note, in the first library CDR1 and CDR2 were modified together while CDR3 remained intact and in the second library only CDR3 was changed. The amplified library was digested with SfiI and BsiWI (NEB, New England Biolabs) and ligated into the pCOMB3X-H(CD24)L(CD24) vector. Ligation reactions were transformed into E. coli strain TG1 and the transformation rate was determined by plating of dilution series. The ligation was performed using a ligation protocol with T4 DNA ligase according to the manufacture's (NEB) instructions. For transformation, 100 μl of the competent cell suspension was mixed with 50-100 ng DNA (ligated or plasmid), in a chilled 13 ml polypropylene tube and incubated on ice for 30 minutes. The cell-plasmid mix was heat-shocked in a water bath at 42° C. for 1 minute and returned to ice for 2 minutes. Following, heat-shock and chilling on ice, 900 μ1 of SOC or LB medium was added to the tube containing competent cells with DNA and incubated at 37° C. with agitation at 250 rpm for 60 minutes. E. coli cells were plated onto LB agar plates supplemented with the appropriate antibiotics and incubated at 37° C. overnight to develop colonies of the transformed cells.

The total number of transformants was 1.45×10⁷ cfu. Total cells from the transformation plates were harvested for plasmid preparation. Affinity maturation was performed in two steps: CDR walking (two steps selection) and phage display technique (described in Antibody Engineering, chapter 38, pp 540-545, 2001).

Panning cycles were performed on each CDR library and good potential binders were found.

Following, the output of each library was combined to create the final combined library as follows :

(1) Each DNA library was digested with PstI and NotI (NEB) and subsequently ligated.

(2) The ligation products were purified by ethanol precipitation and the resulting vectors were introduced by electroporation into XL-1 electro-competent cells (homemade competent cells.

This produced ˜10⁶ individual clones that were ready for rescue with a helper phage (M13KO7) in order to create the final library of phage antibodies for affinity selection.

Following, 4 panning cycles were performed, using stringent pressure selection conditions (Table 6 hereinbelow):

TABLE 6
Pressure selection conditions

| | Input | Output | Selection pressure |
|---|---|---|---|
| Cycle 1 | 10¹² | 10⁶ | (1) 5 washes with PBST and 5 washes with |
| | | | PBS |
| | | | (2) 5 washes with PBST and 5 washes with |
| | | | PBS-5 min between each wash |
| | | | (3) 5 washes with PBST and 5 washes with |
| | | | PBS-10 min between each wash |
| Cycle 2 | 10¹¹ | 5 × 10⁶ | (1) 5 washes with PBST and 5 washes with |
| | | | PBS-10 min between each wash |
| Cycle 3 | 10¹¹ | 5.2 × 10⁶ | (1) 5 washes with PBST and 5 washes with |
| | | | PBS-10 min between each wash |
| | | | (2) 5 washes with PBST and 5 washes with |
| | | | PBS-20 min between each wash |
| | | | (3) 2-fold reduction in antigen amount |
| Cycle 4 | 10¹¹ | 1 × 10⁶ | (1) 5 washes with PBST and 5 washes with |
| | | | PBS-20 min between each wash |
| | | | (2) Blocking with 1% gelatin |

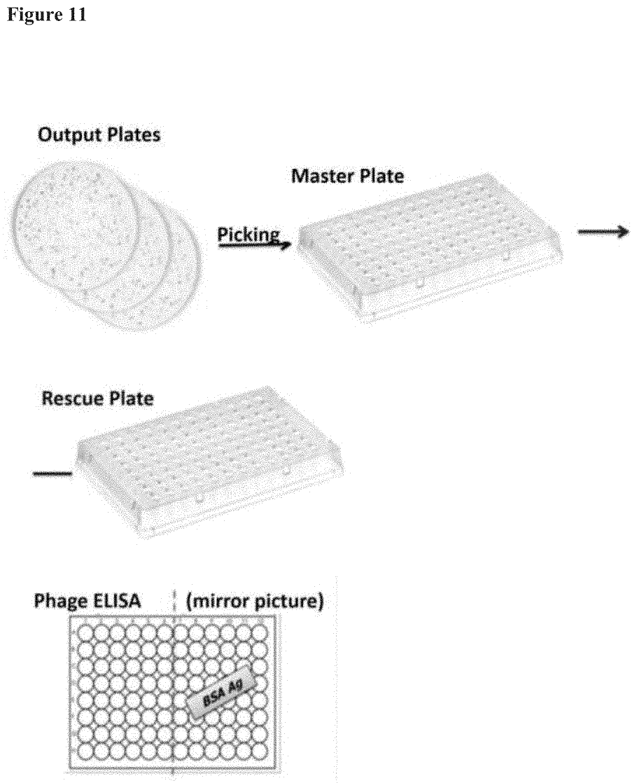

Following the third and fourth panning cycles, phage ELISA was performed in order to evaluate potential binders for antigen binding and specificity. Briefly, individual colonies were picked in sterile 96-wells plate (Master plate) and grew overnight at 37° C. Then, 10 μl from each well were transferred to a second 96-wells plate (Rescue plate) and the rescued phages were evaluated for CD24 binding using ELISA (FIG. 11). Since affinity selection often results in nonspecific phages being isolated along with the specific ones, binding to an irrelevant antigen (BSA) was tested in parallel. The master plates were used as the source for the positive monoclonal clones that were identified by the phage ELISA for further manipulation.

Selected positive clones were validated again by phage ELISA and by soluble Fab ELISA.

DNA from the selected verified clones was isolated and sent to sequencing to determine DNA sequences of the new anti-CD24 antibody mature derivatives.

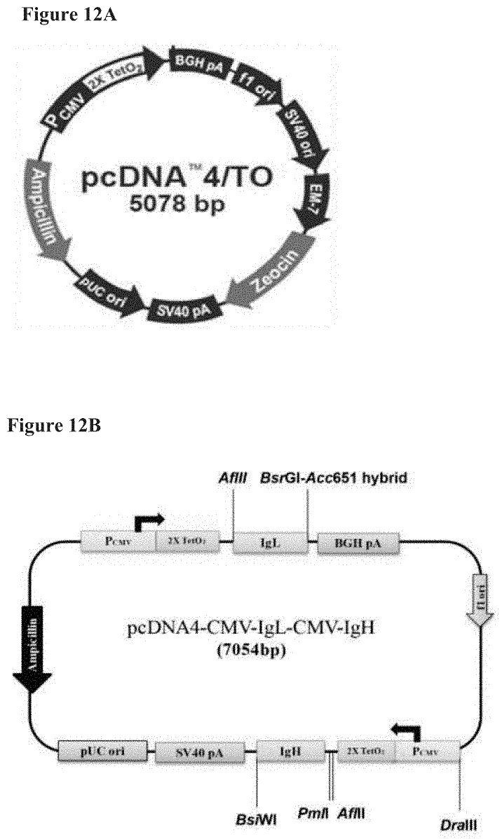

In the next step, the present inventors have constructed an anti-CD24 humanized matured mAbs expression vector for inducible expression and secretion system (FIGS. 12A-B). To this end, the humanized IgH (VH and CH1-CH3 regions) was amplified from pMAZ-IgH(HuSWA11) (SEQ ID NO: 11) (Table 1 hereinabove) and introduced into the pcDNA4/TO vector as a Pm1I/BsiWI fragment. The resulting intermediate vector was named pcDNA4-Hu-IgH. The humanized IgL (regions; VK and CK) was amplified from pMAZ-IgL(HuSWA11) (SEQ ID NO: 12) (Table 1 hereinabove) and introduced into the pcDNA4-Hu-IgH intermediate vector that was digested with Af1II and Acc65I enzymes (NEB), as a Af1II/BsrGI fragment. The resulting vector was named pcDNA4-TOR-CMV-IgL-SV40-IgH. Next, to replace the SV40 promoter with PCMV-TetO2, the PCMV-TetO2 was amplified from pcDNA4-TOR-CMV-IgL-SV40-IgH. The amplified product was introduced into the same vector as DraIII/Pm1I fragment. The resulting vector was named pcDNA4-PCMV-TetO2-IgL-PCMV-TetO2-IgH (SEQ ID NO: 28). The sequences of the variable region which has undergone the maturation procedure were sent for optimization process (IDT) for expression in mammalian cells. It is thought that optimal codons help to achieve faster translation rates and high accuracy. As a result of these factors, translational selection is expected to be stronger in highly expressed genes. The resulted synthesized fragments were digested with EcoRV+BlpI (NEB) and ligated into the pcDNA4-PCMV-TetO2-IgL-PCMV-TetO2 -IgH plasmid that was cleaved with the same enzymes. The sequences of the resulted plasmids were verified by sequencing in the sequencing unit at Tel-Aviv University.

Following, the ability the described novel system to express and secrete the matured anti-CD24 derivatives and the ability of the resulting mAbs to recognize and specifically bind to the CD24 antigen, was evaluated. To this end, supernatant-containing Abs were purified by protein A Sepharose (Amersham Biosciences) chromatography. Briefly, culture supernatant was diluted 1:20 with loading buffer ×20 and loaded onto a protein A column at a flow rate of 0.5 ml/min. The column was extensively washed with loading buffer. Bound antibodies were eluted with 0.1 M of citric acid (pH 3.0) and neutralized with 1 M Tris/HCl (pH 9.0). Protein-containing fractions were combined, dialyzed against 2 L PBS (16 hours at 4° C.), sterile filtered and stored at -20° C.

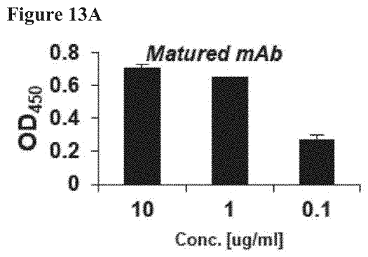

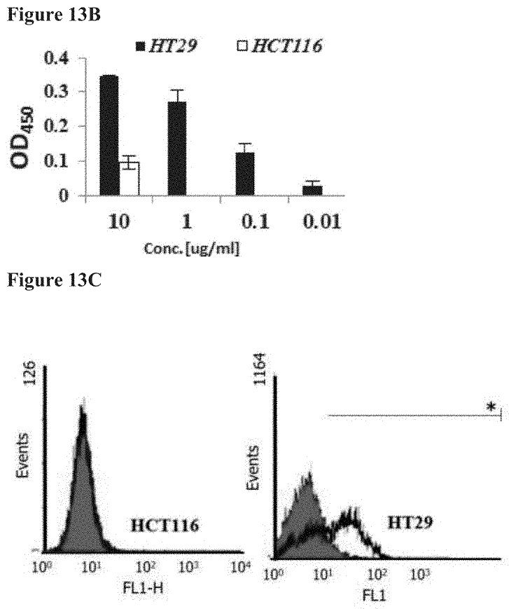

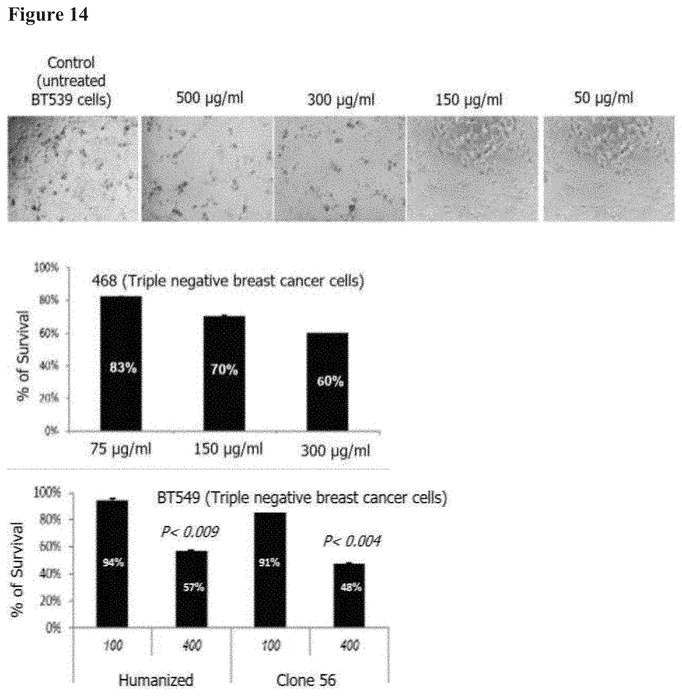

FIGS. 13A-C show a representative selected potential candidate by antigen-based ELISA, whole cell ELISA and FACS analysis. Cell growth comparative growth inhibition analysis was performed on different types of tumor cells, among them triple-negative breast cancer, colorectal, bladder, pancreatic and neuroblastoma cell lines expressing CD24. FIG. 14 shows representative results.

Subsequently, 293T-REx cells were co-transfected with mammalian pcDNA4-TetO2-CMV-IgL-TetO2-CMV-IgH and pEGFP expression vectors using the calcium phosphate procedure. Briefly, 10⁶ cells were seeded into 6-wells plates and 48 hours following transfection, limiting dilutions were performed into 10 cm plates containing 1.2 mg/ml of G418 in growth medium. Stable transfectant cells expressing GFP were identified and detected by fluorescence microscopy and further analyzed for expression and secretion of the humanized Ab. Supernatants of clones growing on medium containing the selection marker were tested for IgG secretion: First, the stable clones were screened by fluorescent microscopy according to the green fluorescence intensity. Clones that expressed the highest levels of the GFP protein were chosen for further evaluation of their ability to secrete these antibodies and the ability of the Abs to recognize and specifically bind to the CD24 antigen.

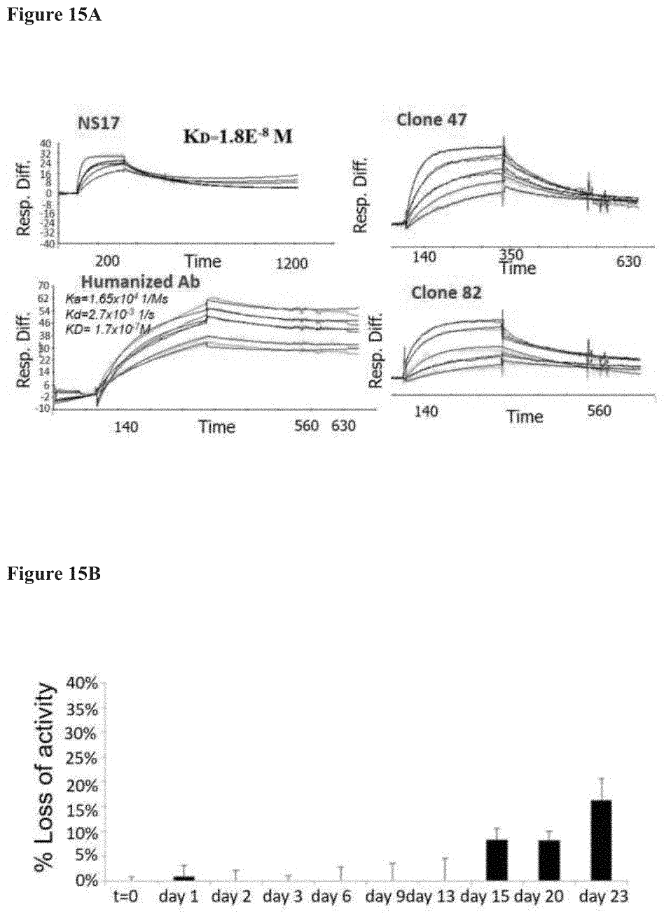

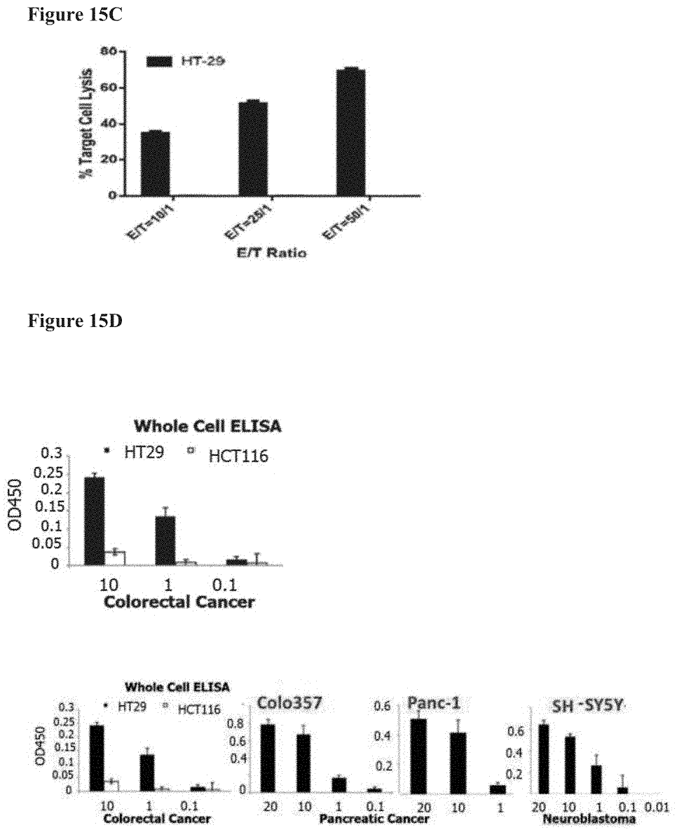

For each matured antibody the best clone was chosen for expression and secretion. A total of eight potential matured antibodies were selected. All of them were expanded for large scale production in 293T-REx cells and purified by protein A column. The purified Ab from each clone was characterized for its ability to specifically recognize and bind the CD24 antigen, binding strength, stability and its ability to induce ADCC and CDC and pharmacokinetic properties (FIGS. 15A-D and Table 7 hereinbelow).

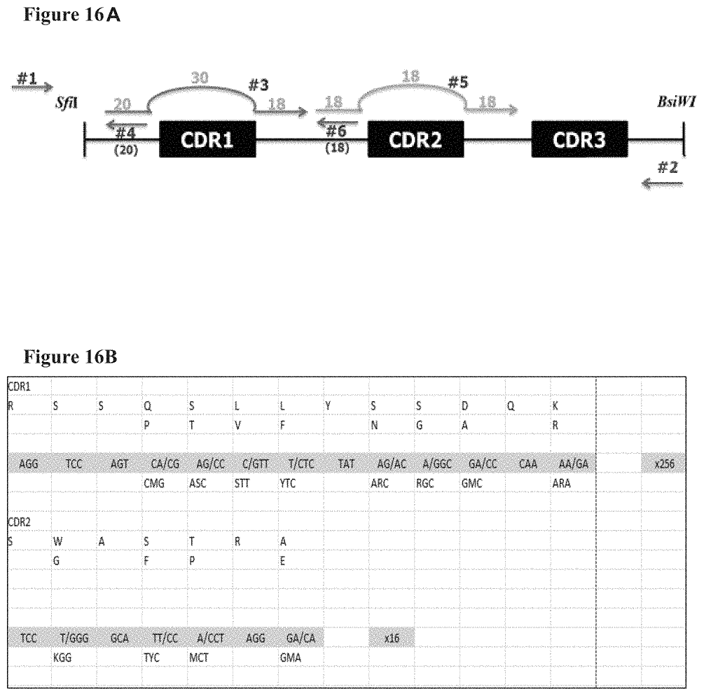

In addition, an additional library was designed based on the sequences of the binders that were isolated in the first maturation process in order to find additional and possibly better binders. For that purpose, the sequences of the parental humanized Ab were arranged against the eight matured derivatives. Following, a multiple sequence alignment was performed using the MULTALIN software in order to find the positions defined as “hot spots” (data not shown). Next, an additional library that comprises all the changes in these loci but discarding changes that caused the loss of binding ability, was designed (FIG. 16A).

Taking into account these considerations, a library that consisted of CDR1 and CDR2 mutations was designed. The library size was 4096 and is illustrated in FIG. 16B. Briefly, DNA of phagemid pComb-Humanized Ab was used as a template in two PCR reactions using primer pairs 3+2 and 1+4 (Table 4 hereinabove), in order to change the VL CDR1 sequence. The PCR products were combined and assembled in a second PCR reaction using primers 1+2 (Table 4 hereinabove). The assembled fragment was used as a template for a subsequent PCR reaction using primer pairs 1+6 (Table 4 hereinabove) and DNA of phagemid pComb-Humanized Ab was used as a template in PCR reaction using primer pairs 2+5 (Table 4 hereinabove) that replaced the VL CDR2 sequence. In the same way, the PCR products were combined and assembled by PCR using primers 1+2 (Table 4 hereinabove).

The resulting library was digested with Sfil+BsiWI restriction enzymes and ligated to pComb-Humanized Ab plasmid that was cleaved with the same enzymes. The ligation product was then purified by ethanol precipitation and transformed into XL-1 electrocompetent cells.

Following, the resulting library was evaluated for potential binders. Individual colonies from the ligation plates were picked and used for ELISA. The results showed that the new library did not contain better binders (data not shown).

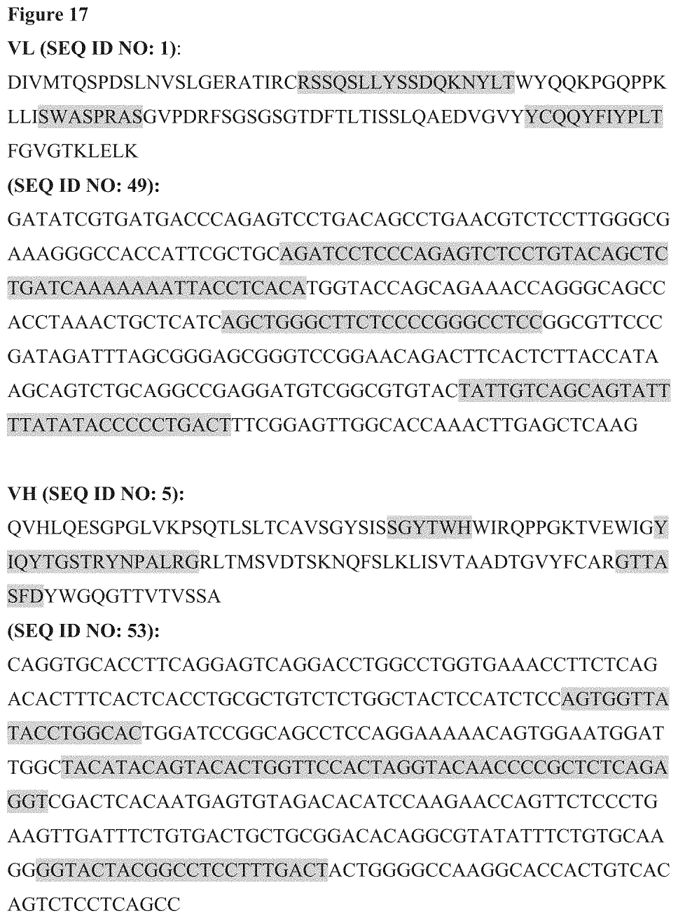

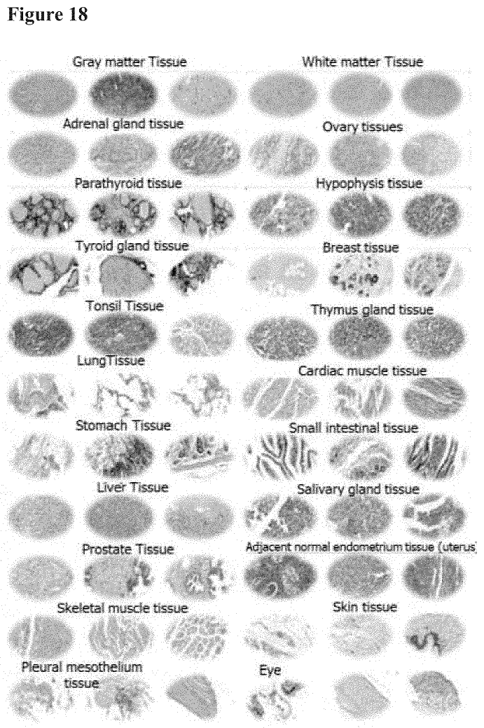

Based on the results presented hereinabove, a single purified humanized mature anti-CD24 antibody, referred to herein as NS17, was selected for further analysis due to its binding affinity, selectivity and stability. The VH and VL sequences of the NS17 antibody are shown in FIG. 17.

TABLE 7
Results of the pharmacokinetic (PK) studies for the matured
antibody NS17 as compared to HuNS17
PK parameters

| | NS17 | HuNS17 |
|---|---|---|
| AUC | 4002 mcg/ml*h | |
| AUMC | 1396469 mcg/ml*h² | |
| MRT | 348 h (14.5 days) | 9.2 days |
| Tmax | 2 h | 1 h |
| Cmax | 9.7 mcg/ml | |
| Half-life | 215-230 h (8.9-9.58 days) | 5.2 days |
| CL | 1.25 ml/h/kg | |
| Vss | 435 ml/kg | |
| Vbeta | 416 ml/kg | |

*The pharmacokinetic (PK) study was performed in SCID mice as follows; SCID female received a single intravenous dose of 5 mg/kg NS17 (n = 12). Blood samples were collected from the periorbital sinus in the case of terminal time points for PK at the following time points: 15 and 30 min; 60, 75, and 90 min, 8, and 24 h; and 3, 7, 10, 14, 18, 21, and 28 days, and transferred into serum separator tubes. Three mice per group underwent periorbital bleeding for each time point, with 150 μl of blood collected per mouse. The presence of the mAb in the collected serum was evaluated and measured by ELISA.

### Example 3

### Anti-Tumor Activity Of The Affinity Matured Humanized Anti-CD24 Monoclonal Antibody

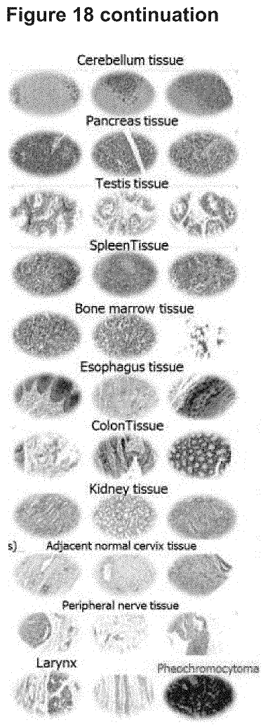

An FDA-approved normal human organ tissue microarray was used in order to characterize the expression pattern of CD24 in healthy human tissues. 32 types of healthy human organs were included based on FDA guidelines, where each organ was taken from 3 healthy human individuals. An anti-CD24 antibody (close SWA11) was used to detect CD24 expression in these tissue. As shown in FIG. 18, the vast majority of the healthy tissues did not express CD24. However, CD24 expression was detected in the developing brain (Pos A2) and in a specific layer of the esophagus tissue.

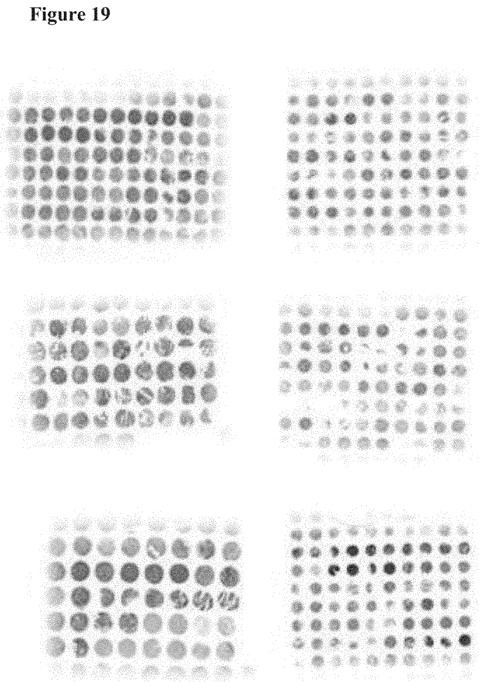

In contrast, several tumor microarrays of various human cancer tissues including adenocarcinoma of the GI tract, bladder, breast, pancreas, RCC, glioblastoma, HCC, NSCLC, ovarian, melanoma indicated that CD24 is highly expressed in most human malignancies (FIG. 19).

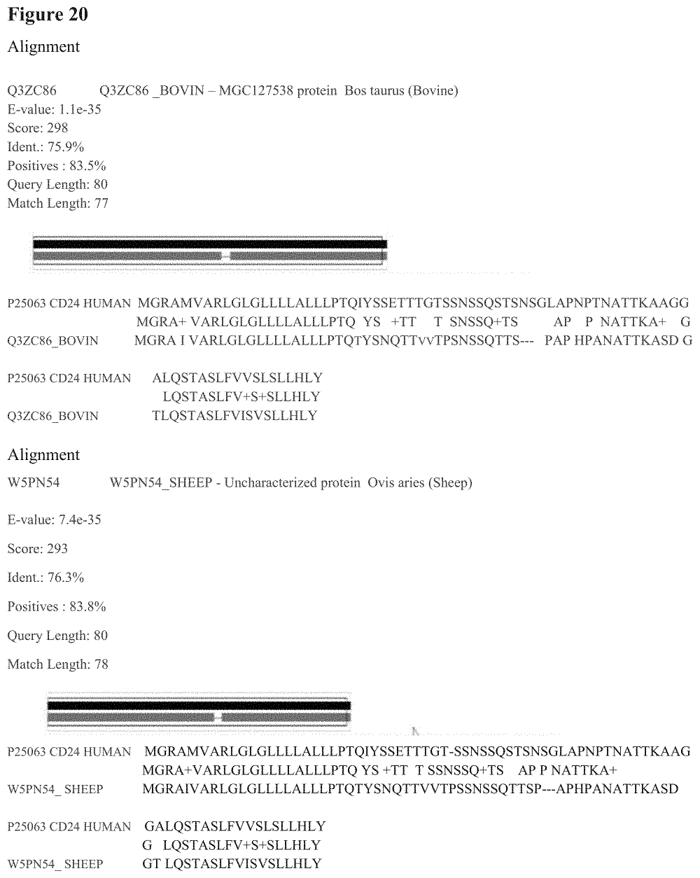

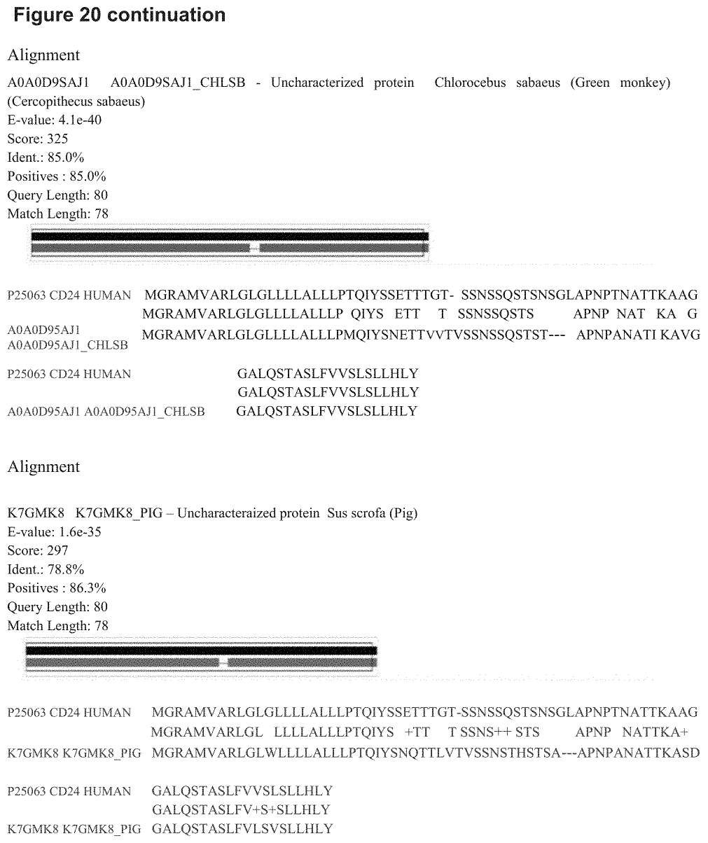

The binding epitope of the SWA11 mAb and the affinity matured humanized NS17 mAb is very unique. A large homology comparison analysis using the Uniprot (www(dot)uniprot(dot)org) was performed. 250 candidates with different percentage of identity were observed. However, in none of them the LAP epitope exists. Some examples are presented in FIG. 20.

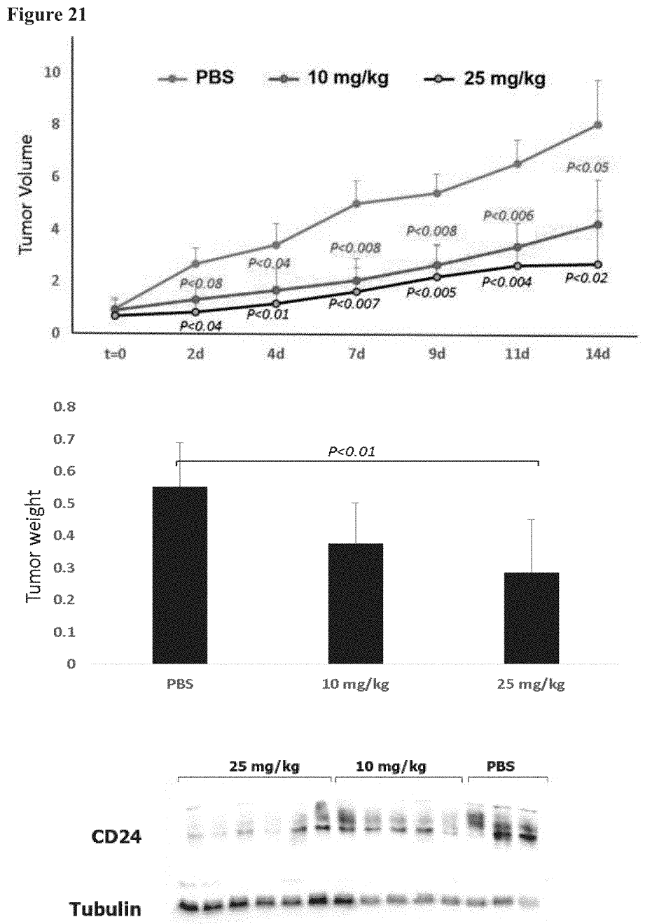

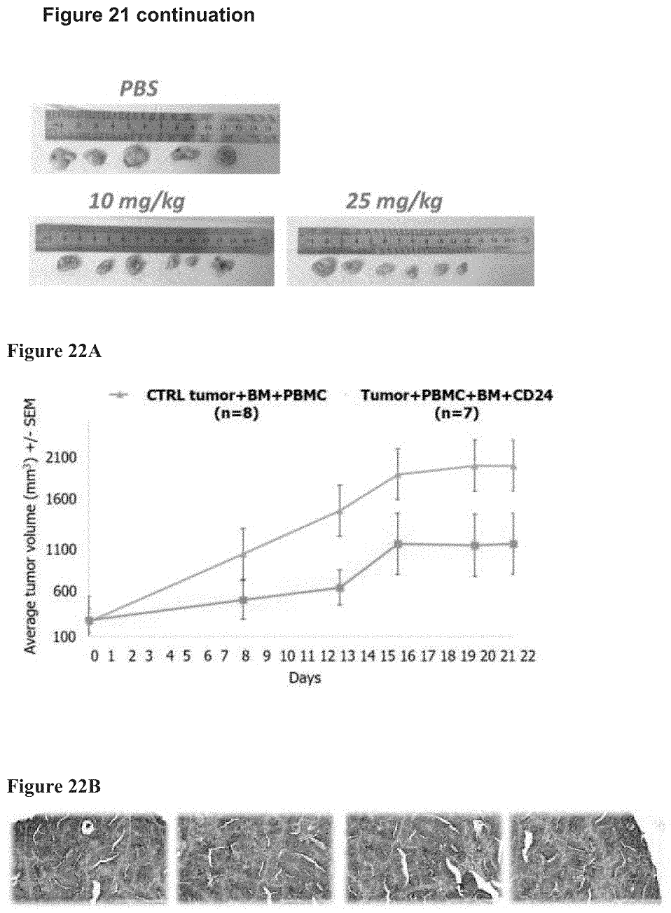

Subsequently, the humanized matured NS17 anti-CD24 antibody produced in CHO cells was tested for its effect on tumor growth in-vivo in a colorectal cancer mouse model. Male 6-8 week old athymic nude mice were housed in sterile cages and handled with aseptic precautions. The mice were fed ad libitum. For testing the therapeutic potential of the mAb (produced in CHO), exponentially growing HT29 cells were harvested and resuspended at a final concentration of 5 x 10⁶ cells per 0.1 ml PBS per injection. The cells were injected subcutaneously at one site on the backs of the mice. When tumors were palpable (˜0.3-0.5 cm³), the mice were randomly divided into three groups and the treatment was initiated. The mAb at two concentrations (10 and 25 mg/kg) or PBS were administrated via intraperitoneal injections with a 3-day interval between injections. The mice were weighed, the tumor volume was measured with a caliper starting from treatment onset, and the results were carefully plotted. Tumor volume was calculated as 4/3π·a·b2. At the end of the experiment, tumors were removed and western blot analysis was performed using the SWA11 murine antibody. As shown in FIG. 21, NS17 inhibited tumor growth in a dose dependent manner. In addition, the downregulation of CD24 expression levels in the tumor cells (due to internalization) confirmed that the antibody indeed reached the tumors.

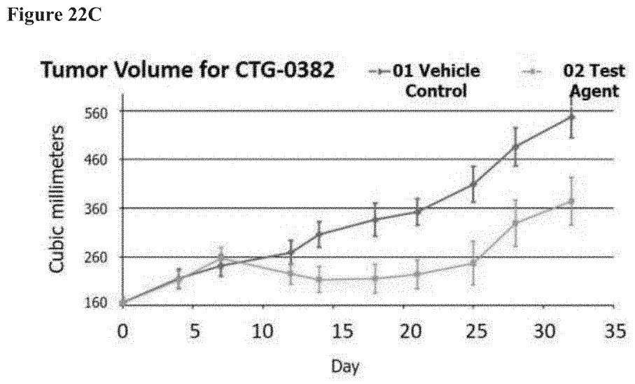

The efficacy of the humanized matured NS17 anti-CD24 antibody was further confirmed using two patient-derived xenograft (PDX) models. In the first experiment (FIG. 22A), bone marrow and tumor tissue samples were taken from a head and neck, Keytruda resistant, male patient. The tumor, passage 3, was transplanted to NGS mice that were irradiated and humanized with the donor stem cells (BM-CD34 HSCs). This study showed that the humanized matured NS17 anti-CD24 antibody reduced tumor volume (by >50%) compared to the control group when administered at a dose of 1 mg/kg via i.v injection (FIG. 22A).

A second experimental protocol (FIGS. 22C) was effected with Champion Oncology. This time the donor had a primary poorly differentiated colorectal adenocarcinoma in the sigmoid (FIG. 22B). This poorly differentiated tumor expressed high levels of CD24 and thus was chosen as the donor for an additional PDX study (FIG. 22B). The CD34+ human immune cells were isolated from cord blood and were used to humanize female NOG mice sublethally irradiated with 175 cGy whole body irradiation. Animals were monitored via clinical observations for three months of the humanization period. About 150 μl of peripheral blood were removed at week 9 and 12 post CD34+ engraftment to assess humanization using mCD45/huCD45/huCD3/huCD19 markers. FACS analysis was performed (Champion Oncology LTD) to detect those markers using conjugated antibodies purchased from BioLegend. Following, tumor cells from the donor were implanted s.c. to mice once humanization was confirmed at the 12 weeks post engraftment period. When sufficient stock animals reached 1.0-1.5 cm³, tumors were harvested for re-implantation into pre-study animals. Pre-study animals were implanted unilaterally on the left flank of humanized mice with tumor fragments harvested from non-humanized stock animals. Each animal was implanted from a specific passage lot and documented. Pre-study tumor volumes were recorded for each experiment beginning seven to ten days following implantation. When tumors reached an average tumor volume of 80-200 mm³ animals were matched by tumor volume into treatment or control groups and dosing initiated on Day 0.

Significant inhibition of tumor growth was achieved upon treatment with the humanized matured NS17 anti-CD24 antibody at a dose of 10 mg/kg via i.p injection (FIG. 22C).

### Example 4

### Phagosytosis-Mediated Activity Of The Affinity Matured Humanized Anti-CD24 Monoclonal Antibody

### Materials and Methods

Cell Culture—Target cells (Human lymphoma cell HL-60 (ATCC® CCL-240™)) were maintained in corresponding complete growth medium at 37° C./5% CO₂ and regularly sub-cultured. Human lymphoma Nalm6 cells (ATCC® CRL-3273™) were maintained in RPMI1640 complete growth medium at 37° C./5% CO2 according to manufacturer's instructions.

Preparation of human monocyte-derived macrophages—PBMCs were isolated using Lymphoprep™ (Axis-Shield PoC AS, Cat # AS1114547) by density gradient centrifugation method. Following, monocytes were purified using human Pan Monocyte Isolation Kit (MiltenyiBiotec, Cat #130-096-537) according to manufacturer's instruction. To prepare Monocytes Derived Macrophages (MDM), monocytes were seeded at a concentration of 5×10⁵ cells/ml in cell culture media, and differentiated to MDM by Macrophage Colony-Stimulating Factor (M-CSF). Specific markers CD11b, CD14, CD45, CD64, CD163 and CD206 on the MDMs were verified by flow cytometry.

Flow cytometry for detection of CD24—Following trypsinization, approximately 5.5×10⁴ cells were washed in fluorescence-activated cell sorting buffer. Following, 20 μg/ml anti-CD24 antibody was added for 60 minutes at 4° C. followed by washing X3 with FACS buffer. Fluorescein isothiocyanate (FITC)—labeled Sheep Anti-Human IgG (FITC) antibody (20 μg/ml) was added for 30 minutes at 4° C. in the dark. Detection of bound antibodies was performed and analyzed using flow cytometer ((BD FACS Calibur, data analysis using FlowJo 7.6.1).

Phagocytosis assay—Human Lymphoma Nalm6 cells were co-cultured with MDMs and treated with the generated humanized mature NS17 anti-CD24 antibody. U5F9-G4 (anti-CD47 mAb) and human IgG were used as a positive and negative controls, respectively. 5-fold dilution with 6 doses of each mAb were used. To quantify antibody-dependent cellular phagocytosis (ADCP) effect, PKH26 (Sigma-aldrich, MINI26-1KT) was used to stain the target Nalm6 cells and APC-anti CD11b antibody (Milteny Biotech, 130-091-241) was used to label MDMs. Cells positive by flow cytometry analysis (BD FACS Calibur followed by data analysis using FlowJo 7.6) for both PKH26 and CD11b-APC were regarded as Target cells-containing MDMs, where phagocytosis took place. Percentage phagocytosis was calculated as the number of the double positive cells against the number of all PKH26 positive cells.

### Results

Antibody-dependent cellular phagocytosis (ADCP) is one of the mechanism of actions of many antibody therapies. It is a highly regulated process by which antibodies eliminate bound targets via connecting their Fc domain to specific receptors on phagocytic cells, and eliciting phagocytosis. To this end the effect of the generated humanized mature NS17 anti-CD24 antibody on phagocytosis events, mediated by human monocyte-derived macrophages (MDM), were analyzed using FACS screening, and a dose dependent curve was generated to assess the ADCP potency in detail.

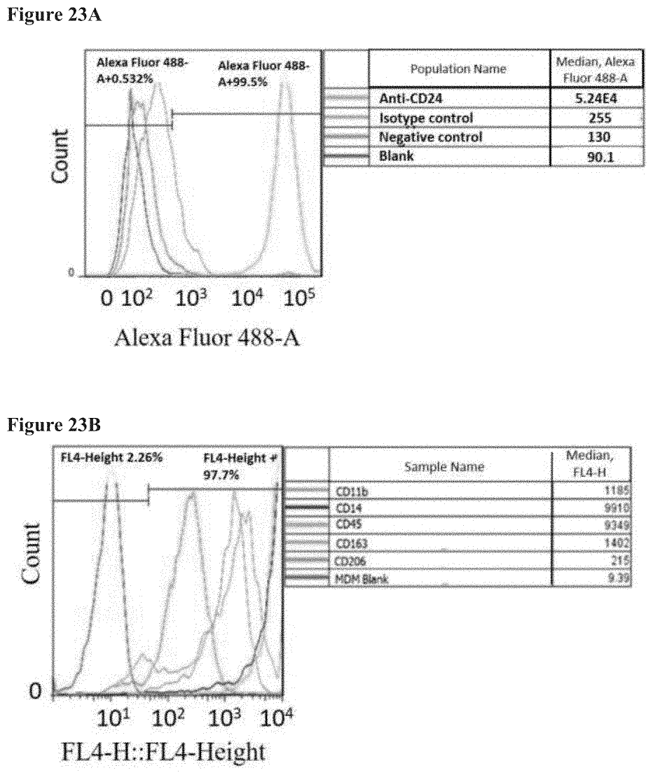

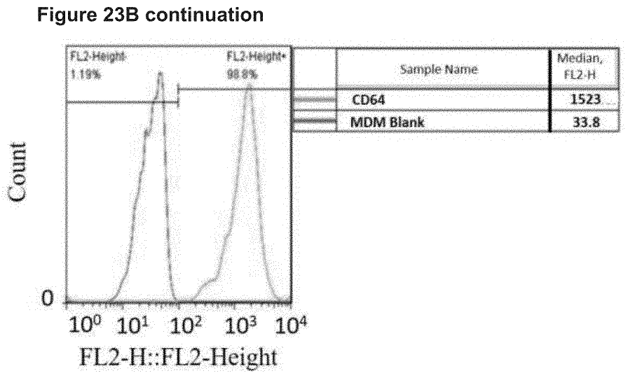

In the first step, expression of CD242 on the target Human Lymphoma Nalm6 cells, and expression of CD11b, CD14, CD45, CD64, CD163 and CD206 on the prepared Monocytes Derived Macrophages (MDM), were verified by flow cytometry (FIGS. 23A-B).

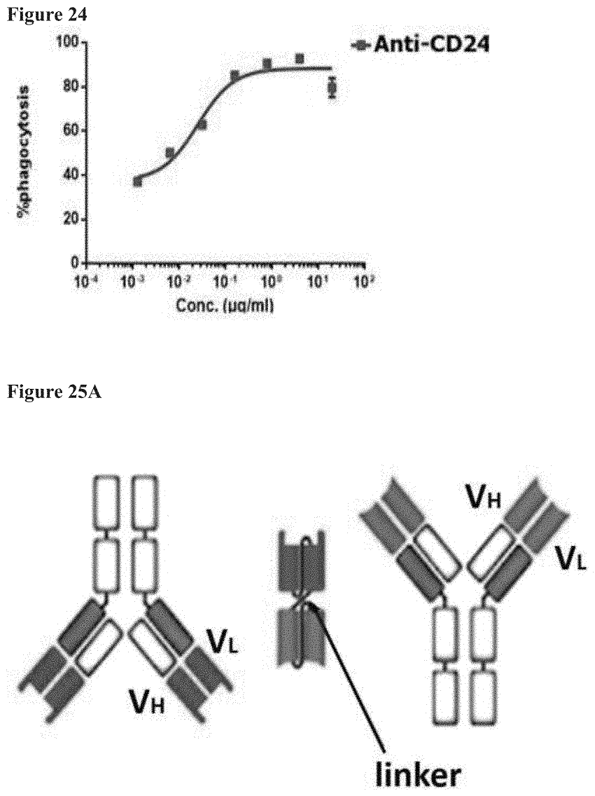

Following, ADCP of Nalm6 cells following treatment with the generated NS17 anti-CD24 was determined. As shown in FIG. 24, the NS17 mAb induced a significant and dose dependent phagocytosis of Nalm6 cells.

### Example 5

### Generation Of A Bispecific Anti-Cd24 Anti-CD3 Antibody

### Materials and Methods

Bispecific T-cell engagers (BiTEs) construction—The sequences of the VL and VH of the humanized mature NS17 and the VL and VH of anti-CD3 (OKT3) were optimized for Homo sapiens (Human). A 5 amino acids or 10 amino acids linker was added between the two arms. Two restriction sites were added, 5′ (EcoRI) and 3′ (XhoI) sequences with sequence verification and the sequences (SEQ ID NO: 30 and 32) were cloned into pcDNA3.4 (Ampicillin) via 5′ EcoRI and 3′ XhoI.

Transfection—BiTEs were transfected to Expi293F cells using ExpiFectamine™ transfection kit (Gibco), according to manufacturer's protocol. In each transfection 7.5×10⁷ cells were added to a 25.5 ml of Expi293 Expression medium in a 125 ml flask. The final volume of each transfection, at the end of the process, was approximately 30 ml. Incubation conditions were 37° C., 8% CO₂ in air, shaken in an orbital shaker at ˜125 rpm. For each transfection a total amount of 30 μg of plasmid DNA

### Transfection Protocol

30 μg of plasmid DNA were diluted in Opti-MEM® I Reduced Serum Medium in a total volume of 1.5 ml.

81 μl of ExpiFectamine 293 Reagent was diluted in Opti-MEM® I medium to a total volume of 1.5 ml. The mixture was incubated for 5 minutes at room temperature.

Following the 5 minutes incubation the diluted DNA was added to the diluted ExpiFectamine 293 Reagent to obtain a total volume of 3 ml.

The mixture was incubated for 20 minutes at room temperature and then added to the cells-containing flask.

Following 20 hours of incubation, 150 μl of ExpiFectamine 293 Transfection Enhancer 1 and 1.5 ml of ExpiFectamine 293 Transfection Enhancer 2 were added to each flask.

The cells were harvested 7 days following transfection.

Purification on Ni-NTA column—The BiTE derivatives were purified using HisTrap HP prepacked columns (packed with Ni Sepharose High performance, affinity resin) according to the manufacturer's instructions (GE healthcare). Binding buffer: 20 mM sodium phosphate, 0.5 M NaCl, 5 mM imidazole, pH 7.4. Elution buffer: 20 mM sodium phosphate, 0.5 M NaCl, 500 mM imidazole, pH 7.4.

Flow cytometry analysis—PBMCs were isolated using a density gradient centrifugation method using Ficoll Histopaque (Sigma-aldrich). 1×106 cells were washed in fluorescence-activated cell sorting buffer. Following, 30 μg/ml BiTE (SEQ ID NO: 29 or SEQ ID NO: 31) antibody was added for 60 minutes at 4° C. followed by washing X3 with FACS buffer. Secondary anti-HIS antibody (20 μg/ml) was added for 30 minutes at 4° C. in the dark. Detection of bound antibodies was performed and analyzed using flow cytometer (BD Cantoll, data analysis using FCS express).

### Results

Bispecific T-cell engagers (BiTEs) are a class of artificial bispecific monoclonal antibodies that form a binding state between e.g. a tumor associated antigen and CD3, to thereby direct T cells activity against e.g. cancer cells independently of the presence of MHC I or co-stimulatory molecules.

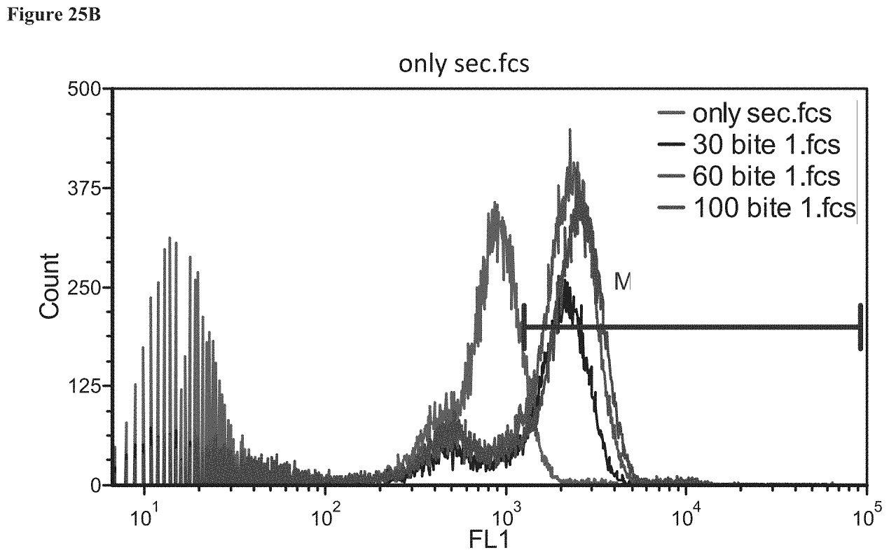

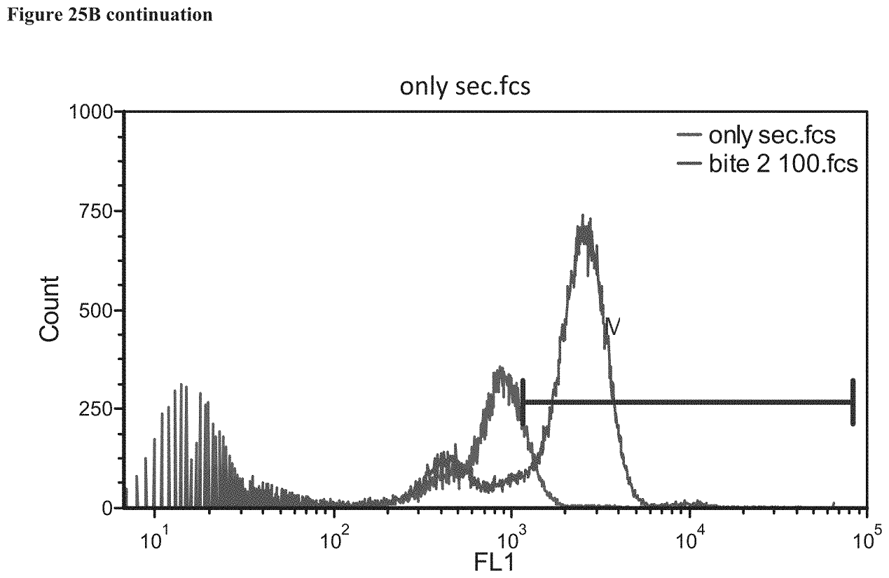

Two BiTE molecules comprising the VH and VL of NS17 and the VH and VL of an anti-CD3 antibody were designed and generated, which differ in the length of the linker between the two arms (FIG. 25A). The first (SEQ ID NO: 29) has a 5 amino acids linker and the second (SEQ ID NO: 31) has a 10 amino acids linker.

The sequences encoding the two BiTEs (SEQ ID NO: 30 or 32, respectively) were cloned into pcDNA3.4 Vector, expressed in Expi293 cells and purified by affinity columns. The ability of the purified BiTEs to bind CD3+CD24+PBMCs was verified by flow cytometry (FIG. 25B).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. An antibody comprising an antigen recognition domain which specifically binds CD24 and comprises complementarity determining regions (CDRs) as set forth in SEQ ID NOs: 2, 3 and 4 arranged in a sequential order from N to C on a light chain of the antibody and CDRs as set forth in SEQ ID NOs: 6, 7 and 8 arranged in a sequential order from N to C on a heavy chain of said antibody.

2. The antibody of claim 1, wherein said light chain amino acid sequence comprises an amino acid sequence as set forth in SEQ ID NO: 1.

3. The antibody of claim 1, wherein said heavy chain amino acid sequence comprises an amino acid sequence as set forth in SEQ ID NO: 5.

4. The antibody of claim 1, wherein said light chain amino acid sequence comprises an amino acid sequence as set forth in SEQ ID NO: 1 and said heavy chain amino acid sequence comprises an amino acid sequence as set forth in SEQ ID NO: 5.

5. The antibody of claim 1, wherein said antibody is a humanized antibody.

6. The antibody of claim 1, wherein said antibody is multispecific.

7. The antibody of claim 6, wherein said antibody is bispecific.

8. The antibody of claim 1, wherein said antibody comprises an antigen recognition domain which specifically binds an immune cell.

9. The antibody of claim 8, wherein said antigen recognition domain which specifically binds said immune cell binds CD3.

10. A polynucleotide encoding the antibody of claim 1.

11. A host cell expressing the antibody of claim 1.

12. A method of producing an anti-CD24 antibody, the method comprising:
(a) culturing the cells of claim 11 under conditions which support production of said antibody; and
(b) recovering said antibody.

13. A method of treating a disease associated with cells expressing CD24 in a subject in need thereof, the method comprising administering to the subject a therapeutically effective amount of the antibody of claim 1, thereby treating the disease in the subject.

14. The method of claim 13, further comprising administering to the subject a therapy for treating said disease.

15. An article of manufacture comprising the antibody of claim 1 and a therapy for treating a disease associated with cells expressing CD24.

16. The method of claim 13, wherein said disease is cancer.

17. The method of claim 16, wherein said cancer is selected from the group consisting of colorectal cancer, pancreatic cancer, B-cell lymphomas, glioma, small-cell lung cancer, non-small cell lung, hepatic cancer, renal cancer, nasopharyngeal cancer, bladder cancer, uterine cancer, ovarian cancer, breast cancer and prostate cancer.

18. The method of claim 16, wherein said cancer is colorectal cancer or pancreatic cancer.

19. The method of claim 16, wherein said therapy is selected from the group consisting of immunotherapy, chemotherapy and radiotherapy.

20. A method of activating an immune cell towards a cell expressing CD24 in a subject in need thereof, the method comprising administering to the subject a therapeutically effective amount of the antibody of claim 8, thereby activating the immune cell towards the cell expressing the CD24.


SEQUENCE LISTING (74 sequences)

SEQ ID NO: 1 - protein, 113 aa, Artificial sequence
  ANTI-CD24 light chain amino acid sequence
DIVMTQSPDSLNVSLGERATIRCRSSQSLLYSSDQKNYLTWYQQKPGQPPKLLISWASPRASGVPDRFSGSGSGTDFTLTISSLQAEDVGVYYCQQYFIYPLTFGVGTKLELK

SEQ ID NO: 2 - protein, 17 aa, Artificial sequence
  complementarity determining regions (CDRs) amino acid sequence
RSSQSLLYSSDQKNYLT

SEQ ID NO: 3 - protein, 8 aa, Artificial sequence
  complementarity determining regions (CDRs) amino acid sequence
SWASPRAS

SEQ ID NO: 4 - protein, 11 aa, Artificial sequence
  complementarity determining regions (CDRs) amino acid sequence
YCQQYFIYPLT

SEQ ID NO: 5 - protein, 118 aa, Artificial sequence
  ANTI-CD24 heavy chain amino acid sequence
QVHLQESGPGLVKPSQTLSLTCAVSGYSISSGYTWHWIRQPPGKTVEWIGYIQYTGSTRYNPALRGRLTMSVDTSKNQFSLKLISVTAADTGVYFCARGTTASFDYWGQGTTVTVSSA

SEQ ID NO: 6 - protein, 6 aa, Artificial sequence
  complementarity determining regions (CDRs) amino acid sequence
SGYTWH

SEQ ID NO: 7 - protein, 16 aa, Artificial sequence
  complementarity determining regions (CDRs) amino acid sequence
YIQYTGSTRYNPALRG

SEQ ID NO: 8 - protein, 7 aa, Artificial sequence
  complementarity determining regions (CDRs) amino acid sequence
GTTASFD

SEQ ID NO: 9 - DNA, 6132 bp, Artificial sequence
  pMAZ-IgH(chSWA11)
gtaccgaattcacattgattattgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacggttagtcatcgctattaccatagtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctttctggctaactagagaacccactgcttactggcacgtggaaattaatacgacgtggccaccatgggatggagctgtatcatcctcttcttggtagcaacagctacaggtaaggggttaacagtagcaggcttgaggtctggacatatatatgggtgacaatgacatccactttgcctttctctccacaggcgcgcactccgatgtgcaccttcaggagtcaggacctgacctggtgaaaccttctcagtcactttcactcacctgcactgtcactggctactccatcaccagtggttatacctggcactggatccggcagtttccaggaaacacagtggaatggatgggctacatacagtacactggttccactaggtacaaccccgctctcagaggtcgactctctatcagtcgagacacatccaagaaccagttcttcctgcagttgatttctgtgactactgcggacacaggcacatatttctgtgcaaggggtactacggcctcctttgactactggggccaaggcaccactctcacagtcgcctcagccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggagaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgatctagaagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggcccgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctctcccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgcccctaggctgactaattttttttatttatgcagaggccgaggccgcctctgcctctgagctattccagaagtagtgaggaggcttttttggaggcctaggcttttgcaaaaagctccccccccgggaggtccacaatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgtatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgatctttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagagtcgttcgctccaagctgggctgtatgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgggcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcagatcgacggatcgggagatcg

SEQ ID NO: 10 - DNA, 6091 bp, Artificial sequence
  pMAZ-IgL(chSWA11)
gtaccgaattcacattgattattgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacggttagtcatcgctattaccatagtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctttctggctaactagagaacccactgcttactggcacgtggaaattaatacgacgtggccaccatgggatggagctgtatcatcctcttcttggtagcaacagctacaggtaaggggttaacagtagcaggcttgaggtctggacatatatatgggtgacaatgacatccactttgcctttctctccacaggcgcgcactccgatatcgtgatgtcacagtctccatcctccctaaatgtgtcagttggagagaaggttactatgaggtgcaggtccagtcagagccttttatatagtagcgatcaaaagaactacttgacctggtaccagcagaaacctgggcagtctcctaaattgctgatttcctgggcatccactagggcatctggggtccctgatcgcttcacaggcagtggatctgggacagatttcactctcaccatcagcagtgtgaaggctgaagacctgggagtttattactgtcaacaatattttatctatccgctcacgttcggtgttgggaccaagctggggctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataatctagagggcccgttttttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggccatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcagatcgacggatcgggagatcg

SEQ ID NO: 11 - DNA, 6132 bp, Artificial sequence
  pMAZ-IgH(HuSWA11)
gtaccgaattcacattgattattgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacggttagtcatcgctattaccatagtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctttctggctaactagagaacccactgcttactggcacgtggaaattaatacgacgtggccaccatgggatggagctgtatcatcctcttcttggtagcaacagctacaggtaaggggttaacagtagcaggcttgaggtctggacatatatatgggtgacaatgacatccactttgcctttctctccacaggcgcgcactcccaggtgcaccttcaggagtcaggacctggcctggtgaaaccttctcagacactttcactcacctgcgctgtctctggctactccatctccagtggttatacctggcactggatccggcagcctccaggaaaaacagtggaatggattggctacatacagtacactggttccactaggtacaaccccgctctcagaggtcgactcacaatgagtgtagacacatccaagaaccagttctccctgaagttgatttctgtgactgctgcggacacaggcgtatatttctgtgcaaggggtactacggcctcctttgactactggggccaaggcaccactgtcacagtctcctcagccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggagaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgatctagaagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggcccgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctctcccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgcccctaggctgactaattttttttatttatgcagaggccgaggccgcctctgcctctgagctattccagaagtagtgaggaggcttttttggaggcctaggcttttgcaaaaagctccccccccgggaggtccacaatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgtatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgatctttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagagtcgttcgctccaagctgggctgtatgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgggcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcagatcgacggatcgggagatcg

SEQ ID NO: 12 - DNA, 6151 bp, Artificial sequence
  pMAZ-IgL(HuSWA11)
gtaccgaattcacattgattattgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacggttagtcatcgctattaccatagtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctttctggctaactagagaacccactgcttactggcacgtggaaattaatacgacgtggccaccatgggatggagctgtatcatcctcttcttggtagcaacagctacaggtaaggggttaacagtagcaggcttgaggtctggacatatatatgggtgacaatgacatccactttgcctttctctccacaggcgcgcactccgatatcgtgatgggtgacaatgacatccactttgcctttctctccacaggcgcgcactccgatatcgtgatgacacagtctccagactccctaaatgtgtcacttggagagagggctactatcaggtgcaggtccagtcagagccttttatatagtagcgatcaaaagaactacttgacctggtaccagcagaaacctgggcagcctcctaaattgctgatttcctgggcatccactagggcatctggggtccctgatcgcttctcaggcagtggatctgggacagatttcactctcaccatcagcagtctgcaggctgaagacgtgggagtttattactgtcaacaatattttatctatccgctcacgttcggtgttgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataatctagagggcccgttttttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggccatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcagatcgacggatcgggagatcg

SEQ ID NO: 13 - DNA, 7049 bp, Artificial sequence
  pcDNA4-TetO2-CMV-IgL-TetO2-CMV-IgH
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaaggccaccatgggatggagctgtatcatcctcttcttggtagcaacagctacaggtaaggggttaacagtagcaggcttgaggtctggacatatatatgggtgacaatgacatccactttgcctttctctccacaggcgcgcactccgatatcgtgatgacacagtctccagactccctaaatgtgtcacttggagagagggctactatcaggtgcaggtccagtcagagccttttatatagtagcgatcaaaagaactacttgacctggtaccagcagaaacctgggcagcctcctaaattgctgatttcctgggcatccactagggcatctggggtccctgatcgcttctcaggcagtggatctgggacagatttcactctcaccatcagcagtctgcaggctgaagacgtgggagtttattactgtcaacaatattttatctatccgctcacgttcggtgttgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaatgtaccgcgggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcacgtgccaccatggagctgtatcatcctcttcttggtagcaacagctacaggtaaggggttaacagtagcaggcttgaggtctggacatatatatgggtgacaatgacatccactttgcctttctctccacaggcgcgcactcccaggtgcaccttcaggagtcaggacctggcctggtgaaaccttctcagacactttcactcacctgcgctgtctctggctactccatctccagtggttatacctggcactggatccggcagcctccaggaaaaacagtggaatggattggctacatacagtacactggttccactaggtacaaccccgctctcagaggtcgactcacaatgagtgtagacacatccaagaaccagttctccctgaagttgatttctgtgactgctgcggacacaggcgtatatttctgtgcaaggggtactacggcctcctttgactactggggccaaggcaccactgtcacagtctcctcagccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgacgtacggatgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 14 - DNA, 6065 bp, Artificial sequence
  pET28a-HIS-Hu-IgL
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggatatcgtgatgacacagtctccagactccctaaatgtgtcacttggagagagggctactatcaggtgcaggtccagtcagagccttttatatagtagcgatcaaaagaactacttgacctggtaccagcagaaacctgggcagcctcctaaattgctgatttcctgggcatccactagggcatctggggtccctgatcgcttctcaggcagtggatctgggacagatttcactctcaccatcagcagtctgcaggctgaagacgtgggagtttattactgtcaacaatattttatctatccgctcacgttcggtgttgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataatctagagggcccgttttttaaaccgctgatcagcctcgactgtgccttctagttgccagccatctgttggaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 15 - DNA, 1976 bp, Artificial sequence
  pCOMB3X-H(CD24)L(CD24)
atgacacagtctccagactccctaaatgtgtcacttggagagagggctactatcaggtgcaggtccagtcagagccttttatatagtagcgatcaaaagaactacttgacctggtaccagcagaaacctgggcagcctcctaaattgctgatttcctgggcatccactagggcatctggggtccctgatcgcttctcaggcagtggatctgggacagatttcactctcaccatcagcagtctgcaggctgaagacgtgggagtttattactgtcaacaatattttatctatccgctcacgttcggtgttgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaatctagataattaattaggaggaatttaaaatgaaatacctattgcctacggcagccgctggattgttattactcgctgcccaaccagccatggcccaggtgcaccttcaggagtcaggacctggcctggtgaaaccttctcagacactttcactcacctgcgctgtctctggctactccatctccagtggttatacctggcactggatccggcagcctccaggaaaaacagtggaatggattggctacatacagtacactggttccactaggtacaaccccgctctcagaggtcgactcacaatgagtgtagacacatccaagaaccagttctccctgaagttgatttctgtgactgctgcggacacaggcgtatatttctgtgcaaggggtactacggcctcctttgactactggggccaaggcaccactgtcacagtctcctcagccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactagtccggactacgcttcttaggagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgtgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaa

SEQ ID NO: 16 - DNA, 1982 bp, Artificial sequence
  pCOMB3X-H(CD30)L(CD24)
atgacacagtctccagactccctaaatgtgtcacttggagagagggctactatcaggtgcaggtccagtcagagccttttatatagtagcgatcaaaagaactacttgacctggtaccagcagaaacctgggcagcctcctaaattgctgatttcctgggcatccactagggcatctggggtccctgatcgcttctcaggcagtggatctgggacagatttcactctcaccatcagcagtctgcaggctgaagacgtgggagtttattactgtcaacaatattttatctatccgctcacgttcggtgttgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataatctagataattaattaggaggaatttaaaatgaaatacctattgcctacggcagccgctggattgttattactcgctgcccaaccagccatggcccaggtccaactgcagcagccggggactgaactggtgaggcctggagcttcagtgaagctgtcctgcaaggcttctggcttctccttcaccagttactggatgaactgggtgaagcagaggcctggacaaggccttgagtggattggcatgattcatccttccgatagtgaaactaggttaaatcagaagttcaaggacagggccacattgactgtagacaaatcctccagcacagcctacatgcaactcagcagcccgacatctgaggactctgcggtctattactgtgcaagtgagatggattattactttgctatggactactggggtcaaggaacctcagtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactagtccggactacgcttcttaggagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgtgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaa

SEQ ID NO: 17 - DNA, 1979 bp, Artificial sequence
  pCOMB3X-H(CD24)L(CD30)
gacattgtgctgacccaatctccaacttctttggctgtgtctctagggcagagggccaccatatcctgcagagccagtgaaagtgttgatagttatggcaatagttttatgcactggttccagcagaaaccaggacagccacccaaactcctcatctatcgtgcatccaacctagaatctgggatccctgccaggttcagtggcagtgggtcttggacagacttcaccctcaccattaatcctgtggaggctgatgatgttgcaacctattactgtcagcaaagtaatgaggatcctcggacgttcggtggaggcaccaaactggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaatctagataattaattaggaggaatttaaaatgaaatacctattgcctacggcagccgctggattgttattactcgctgcccaaccagccatggcccaggtgcaccttcaggagtcaggacctggcctggtgaaaccttctcagacactttcactcacctgcgctgtctctggctactccatctccagtggttatacctggcactggatccggcagcctccaggaaaaacagtggaatggattggctacatacagtacactggttccactaggtacaaccccgctctcagaggtcgactcacaatgagtgtagacacatccaagaaccagttctccctgaagttgatttctgtgactgctgcggacacaggcgtatatttctgtgcaaggggtactacggcctcctttgactactggggccaaggcaccactgtcacagtctcctcagccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactagtccggactacgcttcttaggagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgtgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaa

SEQ ID NO: 18 - DNA, 44 bp, Artificial sequence
  Single strand DNA oligonucleotide
gctaccgtggcccaggcggccgatatcgtgatgacacagtctcc

SEQ ID NO: 19 - DNA, 40 bp, Artificial sequence
  Single strand DNA oligonucleotide
attaattatctagattattaacactctcccctgttgaagc

SEQ ID NO: 20 - DNA, 39 bp, Artificial sequence
  Single strand DNA oligonucleotide
ccaaccagccatggcccaggtgcaccttcaggagtcagg

SEQ ID NO: 21 - DNA, 47 bp, Artificial sequence
  Single strand DNA oligonucleotide
aagcgtagtccggactagttttgtcacaagatttgggctcaactctc

SEQ ID NO: 22 - DNA, 20 bp, Artificial sequence
  Single strand DNA oligonucleotide
cgcgattgcagtggcactgg

SEQ ID NO: 23 - DNA, 22 bp, Artificial sequence
  Single strand DNA oligonucleotide
ggaagatgaagacagatggtgc

SEQ ID NO: 24 - DNA, 68 bp, Artificial sequence
  Single strand DNA oligonucleotide
ctatcaggtgcaggtccagtcmgascsttytctatarcrgcgmccaaaratggtaccagcagaaacct

SEQ ID NO: 25 - DNA, 20 bp, Artificial sequence
  Single strand DNA oligonucleotide
actggacctgcacctgatag

SEQ ID NO: 26 - DNA, 54 bp, Artificial sequence
  Single strand DNA oligonucleotide
cctcctaaattgctgattkgggactycmctagggmaggggtccctgatcgcttc

SEQ ID NO: 27 - DNA, 18 bp, Artificial sequence
  Single strand DNA oligonucleotide
aatcagcaatttaggagg

SEQ ID NO: 28 - DNA, 7049 bp, Artificial sequence
  pcDNA4- PCMV-TetO2 -IgL- PCMV-TetO2 -IgH (NS17)
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaaggccaccatgggatggagctgtatcatcctcttcttggtagcaacagctacaggtaaggggttaacagtagcaggcttgaggtctggacatatatatgggtgacaatgacatccactttgcctttctctccacaggcgcgcactccgatatcgtgatgacccagagtcctgacagcctgaacgtctccttgggcgaaagggccaccattcgctgcagatcctcccagagtctcctgtacagctctgatcaaaaaaattacctcacatggtaccagcagaaaccagggcagccacctaaactgctcatcagctgggcttctccccgggcctccggcgttcccgatagatttagcgggagcgggtccggaacagacttcactcttaccataagcagtctgcaggccgaggatgtcggcgtgtactattgtcagcagtattttatataccccctgactttcggagttggcaccaaacttgagctcaagcgcactgtagccgctccatcagtgttcatctttccccccagcgacgaacagcttaagtctggcacagctagcgttgtgtgcctgctcaacaacttctatcctagggaggctaaggtgcaatggaaggtggataatgcactccagtcaggcaactcccaggaaagtgtaacggaacaggacagcaaggactcaacctactctctctcatccactctgacgctgagcaaagcagactacgagaaacacaaagtctacgcctgcgaagtcacccatcagggcctgagctcgcccgtcacaaagagcttcaacaggggagagtgttaatgtaccgcgggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcacgtgccaccatggagctgtatcatcctcttcttggtagcaacagctacaggtaaggggttaacagtagcaggcttgaggtctggacatatatatgggtgacaatgacatccactttgcctttctctccacaggcgcgcactcccaggtgcaccttcaggagtcaggacctggcctggtgaaaccttctcagacactttcactcacctgcgctgtctctggctactccatctccagtggttatacctggcactggatccggcagcctccaggaaaaacagtggaatggattggctacatacagtacactggttccactaggtacaaccccgctctcagaggtcgactcacaatgagtgtagacacatccaagaaccagttctccctgaagttgatttctgtgactgctgcggacacaggcgtatatttctgtgcaaggggtactacggcctcctttgactactggggccaaggcaccactgtcacagtctcctcagccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgacgtacggatgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 29 - protein, 529 aa, Artificial sequence
  AMINO ACID SEQUENCE OF AN ANTI-CD24 ANTI-CD3 BI-SPECIFIC T CELL ENGAGER (BiTE) COMPRISING A 5 AA LINKER
METGLRWLLLVAVLKGVQCDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSQVHLQESGPGLVKPSQTLSLTCAVSGYSISSGYTWHWIRQPPGKTVEWIGYIQYTGSTRYNPALRGRLTMSVDTSKNQFSLKLISVTAADTGVYFCARGTTASFDYWGQGTTVTVSSAGSAGGGGSGGGGSGGGGSDIVMTQSPDSLNVSLGERATIRCRSSQSLLYSSDQKNYLTWYQQKPGQPPKLLISWASPRASGVPDRFSGSGSGTDFTLTISSLQAEDVGVYYCQQYFIYPLTFGVGTKLELKHHHHHH

SEQ ID NO: 30 - DNA, 1602 bp, Artificial sequence
  NUCLEIC ACID CONSTRUCT ENCODING SEQ ID NO 29
gaattcatggagaccggactgaggtggctgctgctggtcgccgtgctgaagggcgtgcagtgtgatatccaactgacccagtcccccgcctctctggctgtgagcctcggccagagggctacaatcagctgcaaggctagccagtccgtggactacgacggagactcctatctgaattggtatcagcagatccccggccaacctcctaagctgctgatctacgacgcctccaacctcgtcagcggcatccctcctaggtttagcggctccggcagcggcacagacttcacactgaacatccatcccgtcgaaaaggtggatgccgccacatatcactgccagcagagcaccgaggacccttggaccttcggcggaggcacaaagctggagatcaaaggcggaggaggaagcggaggcggaggatccggaggaggaggatcccaagtgcagctccaacagagcggagctgagctcgtcagacccggctccagcgtcaagattagctgcaaggcctccggctacgccttttcctcctactggatgaactgggtgaagcaaaggcccggccaaggactggagtggatcggacagatctggcccggcgatggagacaccaactacaacggcaagttcaagggcaaggccaccctcacagctgatgagtccagctccaccgcctacatgcagctgtcctctctggccagcgaagatagcgctgtctacttctgcgctagaagagagacaaccaccgtcggaagatactactatgccatggattactggggccaaggaaccaccgtcaccgtgtcctccggaggaggaggatcccaagtgcatctgcaagagagcggacccggcctcgtcaagccctcccaaaccctctctctgacatgcgctgtgtccggctattccatttccagcggctacacatggcactggattagacagcctcccggaaagaccgtggaatggatcggctacatccagtacaccggaagcaccagatacaaccccgctctgagaggaagactgaccatgtccgtggacaccagcaaaaaccagttctctctgaagctgatctccgtgaccgctgctgacaccggcgtgtacttttgcgctagaggaacaaccgccagcttcgactattggggacaaggcacaacagtgaccgtctcctccgccggatccgctggcggaggaggctccggcggcggcggcagcggcggaggcggctccgacatcgtgatgacccaaagccccgactctctgaacgtgtctctgggagagagggctaccatcagatgtaggtcctcccagtctctgctgtactcctccgaccagaaaaactacctcacatggtaccagcagaagcccggccaaccccctaaactgctgattagctgggcttcccccagagctagcggagtgcccgatagattttccggcagcggatccggcaccgatttcaccctcaccatctcctccctccaagccgaagatgtcggcgtgtactactgccagcagtatttcatctaccccctcacattcggcgtgggcaccaagctcgaactcaagcaccatcaccatcaccactgactcgag

SEQ ID NO: 31 - protein, 534 aa, Artificial sequence
  AMINO ACID SEQUENCE OF AN ANTI-CD24 ANTI-CD3 BI-SPECIFIC T CELL ENGAGER (BiTE) COMPRISING A 10 AA LINKER
METGLRWLLLVAVLKGVQCDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGGGGSQVHLQESGPGLVKPSQTLSLTCAVSGYSISSGYTWHWIRQPPGKTVEWIGYIQYTGSTRYNPALRGRLTMSVDTSKNQFSLKLISVTAADTGVYFCARGTTASFDYWGQGTTVTVSSAGSAGGGGSGGGGSGGGGSDIVMTQSPDSLNVSLGERATIRCRSSQSLLYSSDQKNYLTWYQQKPGQPPKLLISWASPRASGVPDRFSGSGSGTDFTLTISSLQAEDVGVYYCQQYFIYPLTFGVGTKLELKHHHHHH

SEQ ID NO: 32 - DNA, 1617 bp, Artificial sequence
  NUCLEIC ACID CONSTRUCT ENCODING SEQ ID NO 31
gaattcatggagaccggactgaggtggctgctgctggtcgccgtgctgaagggcgtgcagtgtgatatccaactgacccagtcccccgcctctctggctgtgagcctcggccagagggctacaatcagctgcaaggctagccagtccgtggactacgacggagactcctatctgaattggtatcagcagatccccggccaacctcctaagctgctgatctacgacgcctccaacctcgtcagcggcatccctcctaggtttagcggctccggcagcggcacagacttcacactgaacatccatcccgtcgaaaaggtggatgccgccacatatcactgccagcagagcaccgaggacccttggaccttcggcggaggcacaaagctggagatcaaaggcggaggaggaagcggaggcggaggatccggaggaggaggatcccaagtgcagctccaacagagcggagctgagctcgtcagacccggctccagcgtcaagattagctgcaaggcctccggctacgccttttcctcctactggatgaactgggtgaagcaaaggcccggccaaggactggagtggatcggacagatctggcccggcgatggagacaccaactacaacggcaagttcaagggcaaggccaccctcacagctgatgagtccagctccaccgcctacatgcagctgtcctctctggccagcgaagatagcgctgtctacttctgcgctagaagagagacaaccaccgtcggaagatactactatgccatggattactggggccaaggaaccaccgtcaccgtgtcctccggaggaggaggatccggcggcggcggcagccaagtgcatctgcaagagagcggacccggcctcgtcaagccctcccaaaccctctctctgacatgcgctgtgtccggctattccatttccagcggctacacatggcactggattagacagcctcccggaaagaccgtggaatggatcggctacatccagtacaccggaagcaccagatacaaccccgctctgagaggaagactgaccatgtccgtggacaccagcaaaaaccagttctctctgaagctgatctccgtgaccgctgctgacaccggcgtgtacttttgcgctagaggaacaaccgccagcttcgactattggggacaaggcacaacagtgaccgtctcctccgccggatccgctggcggaggaggctccggcggcggcggcagcggcggaggcggctccgacatcgtgatgacccaaagccccgactctctgaacgtgtctctgggagagagggctaccatcagatgtaggtcctcccagtctctgctgtactcctccgaccagaaaaactacctcacatggtaccagcagaagcccggccaaccccctaaactgctgattagctgggcttcccccagagctagcggagtgcccgatagattttccggcagcggatccggcaccgatttcaccctcaccatctcctccctccaagccgaagatgtcggcgtgtactactgccagcagtatttcatctaccccctcacattcggcgtgggcaccaagctcgaactcaagcaccatcaccatcaccactgactcgag

SEQ ID NO: 33 - DNA, 396 bp, Artificial sequence
  VL ANTI-CD3 nucleic acid sequence
gaattcatggagaccggactgaggtggctgctgctggtcgccgtgctgaagggcgtgcagtgtgatatccaactgacccagtcccccgcctctctggctgtgagcctcggccagagggctacaatcagctgcaaggctagccagtccgtggactacgacggagactcctatctgaattggtatcagcagatccccggccaacctcctaagctgctgatctacgacgcctccaacctcgtcagcggcatccctcctaggtttagcggctccggcagcggcacagacttcacactgaacatccatcccgtcgaaaaggtggatgccgccacatatcactgccagcagagcaccgaggacccttggaccttcggcggaggcacaaagctggagatcaaa

SEQ ID NO: 34 - protein, 130 aa, Artificial sequence
  VL ANTI-CD3 Amino acid sequence
METGLRWLLLVAVLKGVQCDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 35 - protein, 33 aa, Artificial sequence
  complementarity determining regions (CDRs) sequence
CAGTCCGTGGACTACGACGGAGACTCCTATCTG

SEQ ID NO: 36 - protein, 11 aa, Artificial sequence
  complementarity determining regions (CDRs) sequence
QSVDYDGDSYL

SEQ ID NO: 37 - DNA, 9 bp, Artificial sequence
  complementarity determining regions (CDRs) sequence
gacgcctcc

SEQ ID NO: 38 - protein, 3 aa, Artificial sequence
  complementarity determining regions (CDRs) sequence
DAS

SEQ ID NO: 39 - DNA, 27 bp, Artificial sequence
  complementarity determining regions (CDRs) sequence
cagcagagcaccgaggacccttggacc

SEQ ID NO: 40 - protein, 9 aa, Artificial sequence
  complementarity determining regions (CDRs) sequence
QQSTEDPWT

SEQ ID NO: 41 - DNA, 372 bp, Artificial sequence
  41 VH ANTI-CD3 NA
caagtgcagctccaacagagcggagctgagctcgtcagacccggctccagcgtcaagattagctgcaaggcctccggctacgccttttcctcctactggatgaactgggtgaagcaaaggcccggccaaggactggagtggatcggacagatctggcccggcgatggagacaccaactacaacggcaagttcaagggcaaggccaccctcacagctgatgagtccagctccaccgcctacatgcagctgtcctctctggccagcgaagatagcgctgtctacttctgcgctagaagagagacaaccaccgtcggaagatactactatgccatggattactggggccaaggaaccaccgtcaccgtgtcctcc

SEQ ID NO: 42 - protein, 124 aa, Artificial sequence
  VH ANTI-CD3 AA
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 43 - DNA, 24 bp, Artificial sequence
  complementarity determining regions (CDRs) amino acid sequence
ggctacgccttttcctcctactgg

SEQ ID NO: 44 - protein, 8 aa, Artificial sequence
  complementarity determining regions (CDRs) amino acid sequence
GYAFSSYW

SEQ ID NO: 45 - DNA, 24 bp, Artificial sequence
  complementarity determining regions (CDRs) amino acid sequence
atctggcccggcgatggagacacc

SEQ ID NO: 46 - protein, 8 aa, Artificial sequence
  complementarity determining regions (CDRs) amino acid sequence
IWPGDGDT

SEQ ID NO: 47 - DNA, 51 bp, Artificial sequence
  complementarity determining regions (CDRs) amino acid sequence
gctagaagagagacaaccaccgtcggaagatactactatgccatggattac

SEQ ID NO: 48 - protein, 17 aa, Artificial sequence
  complementarity determining regions (CDRs) amino acid sequence
ARRETTTVGRYYYAMDY

SEQ ID NO: 49 - DNA, 339 bp, Artificial sequence
  VL ANTI-CD24 NA
gatatcgtgatgacccagagtcctgacagcctgaacgtctccttgggcgaaagggccaccattcgctgcagatcctcccagagtctcctgtacagctctgatcaaaaaaattacctcacatggtaccagcagaaaccagggcagccacctaaactgctcatcagctgggcttctccccgggcctccggcgttcccgatagatttagcgggagcgggtccggaacagacttcactcttaccataagcagtctgcaggccgaggatgtcggcgtgtactattgtcagcagtattttatataccccctgactttcggagttggcaccaaacttgagctcaag

SEQ ID NO: 50 - DNA, 51 bp, Artificial sequence
  complementarity determining regions (CDRs) amino acid sequence
agatcctcccagagtctcctgtacagctctgatcaaaaaaattacctcaca

SEQ ID NO: 51 - DNA, 24 bp, Artificial sequence
  complementarity determining regions (CDRs) amino acid sequence
agctgggcttctccccgggcctcc

SEQ ID NO: 52 - DNA, 33 bp, Artificial sequence
  complementarity determining regions (CDRs) amino acid sequence
tattgtcagcagtattttatataccccctgact

SEQ ID NO: 53 - DNA, 354 bp, Artificial sequence
  VH ANTI-CD24 NA
caggtgcaccttcaggagtcaggacctggcctggtgaaaccttctcagacactttcactcacctgcgctgtctctggctactccatctccagtggttatacctggcactggatccggcagcctccaggaaaaacagtggaatggattggctacatacagtacactggttccactaggtacaaccccgctctcagaggtcgactcacaatgagtgtagacacatccaagaaccagttctccctgaagttgatttctgtgactgctgcggacacaggcgtatatttctgtgcaaggggtactacggcctcctttgactactggggccaaggcaccactgtcacagtctcctcagcc

SEQ ID NO: 54 - DNA, 18 bp, Artificial sequence
  complementarity determining regions (CDRs) amino acid sequence
agtggttatacctggcac

SEQ ID NO: 55 - DNA, 48 bp, Artificial sequence
  complementarity determining regions (CDRs) amino acid sequence
tacatacagtacactggttccactaggtacaaccccgctctcagaggt

SEQ ID NO: 56 - DNA, 22 bp, Artificial sequence
  complementarity determining regions (CDRs) amino acid sequence
ggtactacggcctcctttgact

SEQ ID NO: 57 - protein, 117 aa, Artificial sequence
  heavy chain V-region of murine SWA11
DVHLQESGPDLVKPSQSLSLTCTVTGYSITSGYSWHWIRQFPGNTVEWMGYIQYTGSTRYNPALRGRLSISRDTSKNQFFLQLISVTTADTGTYFCARGTTASFDYWGQGTTLTVSS

SEQ ID NO: 58 - DNA, 351 bp, Artificial sequence
  heavy chain V-region of murine SWA11
gatgtgcaccttcaggagtcaggacctgacctggtgaaaccttctcagtcactttcactcacctgcactgtcactggctactccatcaccagtggttatacctggcactggatccggcagtttccaggaaacacagtggaatggatgggctacatacagtacactggttccactaggtacaaccccgctctcagaggtcgactctctatcagtcgagacacatccaagaaccagttcttcctgcagttgatttctgtgactactgcggacacaggcacatatttctgtgcaaggggtactacggcctcctttgactactggggccaaggcaccactctcacagtctcctca

SEQ ID NO: 59 - protein, 109 aa, Artificial sequence
  heavy chain V-region of donor sequences from IGVH-28*02 and JH6
QVQLQESGPGLVKPSQTLSLTCAVSGYSISSSNWWGWIRQPPGKGLEWIGYIYYSGSIYYNPSLKSRVTMSVDTSKNQFSLKLSSVTAVDTAVYYCARWGQGTTVTVSS

SEQ ID NO: 60 - protein, 117 aa, Artificial sequence
  heavy chain V-region of donor sequences of humanized SWA11
QVHLQESGPGLVKPSQTLSLTCTVSGYSISSGYSWHWIRQFPGKTVEWIGYIQYTGSTRYNPALRGRLTMSVDTSKNQFSLKLISVTAADTGVYFCARGTTASFDYWGQGTTVTVSS

SEQ ID NO: 61 - DNA, 354 bp, Artificial sequence
  heavy chain V-region of donor sequences of humanized SWA11
caggtgcaccttcaggagtcaggacctggcctggtgaaaccttctcagacactttcactcacctgcactgtctctggctactccatctccagtggttatacctggcactggatccggcagcctccaggaaaaacagtggaatggattggctacatacagtacactggttccactaggtacaaccccgctctcagaggtcgactcacaatgagtgtagacacatccaagaaccagttctccctgaagttgatttctgtgactgctgcggacacaggcgtatatttctgtgcaaggggtactacggcctcctttgactactggggccaaggcaccactgtcacagtcgcctcagcc

SEQ ID NO: 62 - protein, 113 aa, Artificial sequence
  D  I  V  M  S  Q  S  P  S  S  L  N  V  S  V  G E  K  V  T  M  R  C R  S  S  Q  S  L  L  Y  S  S  D  Q  K  N  Y  L T     W  Y  Q  Q  K  P  G Q  S  P  K  L  L  I  S     W  A  S  T  R A  S     G  V  P  D  R  F  T  G S  G  S  G  T  D  F  T  L  T  I  S S  V  K  A
DIVMSQSPSSLNVSVGEKVTMRCRSSQSLLYSSDQKNYLTWYQQKPGQSPKLLISWASTRASGVPDRFTGSGSGTDFTLTISSVKAEDLGVYYCQQYFIYPLTFGVGTKLGLK

SEQ ID NO: 63 - DNA, 338 bp, Artificial sequence
  light chain V-region of murine SWA11
gatatcgtgatgtcacagtctccatcctccctaaatgtgtcagttggagagaaggttactatgaggtgcggtccagtcagagccttttatatagtagcgatcaaaagaactacttgacctggtaccagcagaaacctgggcagtctcctaaattgctgatttcctgggcatccactagggcatctggggtccctgatcgcttcacaggcagtggatctgggacagatttcactctcaccatcagcagtgtgaaggctgaagacctgggagtttattactgtcaacaatattttatctatccgctcacgttcggtgttgggaccaagctggggctgaaa

SEQ ID NO: 64 - protein, 108 aa, Artificial sequence
  light chain V-region of DONORIGK4-1*01 AND JK2
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYFGQGTKLEIK

SEQ ID NO: 65 - protein, 113 aa, Artificial sequence
  light chain V-region of humanized SWA11
DIVMTQSPDSLNVSLGERATIRCRSSQSLLYSSDQKNYLTWYQQKPGQPPKLLISWASTRASGVPDRFSGSGSGTDFTLTISSLQAEDVGVYYCQQYFIYPLTFGVGTKLELK

SEQ ID NO: 66 - DNA, 338 bp, Artificial sequence
  light chain V-region of humanized SWA11
gatatcgtgatgacacagtctccagactccctaaatgtgtcacttggagagagggctactatcaggtgcggtccagtcagagccttttatatagtagcgatcaaaagaactacttgacctggtaccagcagaaacctgggcaccctcctaaattgctgatttcctgggcatccactagggcatctggggtccctgatcgcttctcaggcagtggatctgggacagatttcactctcaccatcagcagtctgcaggctgaagacgtgggagtttattactgtcaacaatattttatctatccgctcacgttcggtgttgggaccaagctggagctgaaa

SEQ ID NO: 67 - protein, 51 aa, Artificial sequence
  complementarity determining regions (CDRs) amino acid sequence
AVRTCCAGTCAGAVYKTRTTNWABARYRRNRHNMVNARRAACTWYTTGRCY

SEQ ID NO: 68 - protein, 24 aa, Artificial sequence
  complementarity determining regions (CDRs) amino acid sequence
TMCKKBGCATYYSMNCGTRMRTCT

SEQ ID NO: 69 - protein, 33 aa, Artificial sequence
  complementarity determining regions (CDRs) amino acid sequence
TWYTGCMANMANNVYHDYMNYHHYYCNYNBTAC

SEQ ID NO: 70 - protein, 79 aa, Artificial sequence
  example of the CD24 amino acid sequence (partial P25063)
MGRAMVARLGLGLLLLALLLPTQIYSSETTTGTSSNSSQSTSNSGLAPNPTNATTKAAGGALQSTASLFVVSLSLLHLY

SEQ ID NO: 71 - protein, 76 aa, Artificial sequence
  example of the CD24 amino acid sequence (partial Q3ZC86)
MGRAIVARLGLGLLLLALLLPTQTYSNQTTVVTPSNSSQTTSPAPHPANATTKASDGTLQSTASLFVISVSLLHLY

SEQ ID NO: 72 - protein, 77 aa, Artificial sequence
  example of the CD24 amino acid sequence (partial W5PN54)
MGRAIVARLGLGLLLLALLLPTQTYSNQTTVVTPSSNSSQTTSPAPHPANATTKASDGTLQSTASLFVISVSLLHLY

SEQ ID NO: 73 - protein, 77 aa, Artificial sequence
  example of the CD24 amino acid sequence (partial A0A0D95AJ1)
MGRAMVARLGLGLLLLALLLPMQIYSNETTVVTVSSNSSQSTSTAPNPANATIKAVGGALQSTASLFVVSLSLLHLY

SEQ ID NO: 74 - protein, 77 aa, Artificial sequence
  example of the CD24 amino acid sequence (partial K7GMK8)
MGRAMVARLGLWLLLLALLLPTQIYSNQTTLVTVSSNSTHSTSAAPNPANATTKASDGALQSTASLFVLSVSLLHLY